(19) United States
(12) Patent Application Publication
(10) Pub. No.: US 2021/0087250 A1
(43) Pub. Date: Mar. 25, 2021

(54) PHARMACEUTICAL COMPOSITIONS FOR THE TRANSMUCOSAL DELIVERY OF THERAPEUTIC PEPTIDES AND PROTEINS

(71) Applicant: CYPRUMED GMBH, Obsteig (AT)

(72) Inventors: Martin WERLE, Bludenz (AT); Florian FÖGER, Obsteig (AT)

(73) Assignee: CYPRUMED GMBH, Obsteig (AT)

(21) Appl. No.: 17/045,535

(22) PCT Filed: Apr. 8, 2019
(86) PCT No.: PCT/EP2019/058828

§ 371 Date: Oct. 6, 2020

(30) Foreign Application Priority Data
Apr. 6, 2018 (EP) 18166177.8
Dec. 10, 2018 (EP) 18211296.1

(51) Int. Cl. C07K 14/62

(52) U.S. Cl. CPC C07K 14/62; A61K 45/06

(57) ABSTRACT

The present invention relates to a pharmaceutical composition for transmucosal administration, comprising a peptide or protein drug in combination with an excipient with a pKₐ value of 12 or higher (e.g., arginine free base, EDTA tetrasodium salt, trisodium phosphate, tris(hydroxymethyl)aminomethane, lysine, or calcium hydroxide).

The present invention relates to a pharmaceutical composition for transmucosal administration, comprising a peptide or protein drug in combination with an excipient with a pKₐ value of 12 or higher (e.g., arginine free base, EDTA tetrasodium salt, trisodium phosphate, tris(hydroxymethyl)aminomethane, lysine, or calcium hydroxide).

There is a strong need for effective, shelf-stable but also safe compositions for the non-invasive delivery of biologics such as peptides and proteins. Besides low permeability across transmucosal membranes (e.g., gastro-intestinal or nasal mucosa) the enzymatic degradation of peptides and proteins is one of the most crucial barriers to successful delivery of such therapeutics. It has been reported long time ago that peptidases like trypsin can be reversibly inactivated at alkaline pH levels (Kunitz M et al. J Gen Physiol. 1934; 17(4):591-615), which also holds true for bacterial peptidases in the intestinal tract. However, it has never been reported that this concept of enzyme inhibition could be applied to pharmaceutical formulations for the transmucosal delivery of therapeutic peptides and proteins. A successful formulation would not just need to generate a pH that is sufficiently high to reduce the enzymatic activity of proteolytic enzymes in close proximity where the dosage form releases the therapeutic peptide or protein, but would also need to do so with very safe excipients that are suitable for pharmaceutical administration. In addition, it is known that peptides and proteins are prone to chemical degradation at alkaline pH (Brange J et al. Acta Pharm Nord. 1992; 4(3):149-58), so formulations would also need to be developed that are sufficiently shelf-stable.

The present invention addresses these shortcomings in the art and provides pharmaceutical compositions that are advantageously stable in the presence of proteolytic enzymes and thus allow a particularly efficient delivery of therapeutic peptides or proteins via the transmucosal route, particularly via the oromucosal route, as also demonstrated in the appended Examples.

Accordingly, the present invention provides a pharmaceutical composition for transmucosal administration, comprising a peptide or protein drug and an excipient with a pKₐ value of 12 or higher.

The invention likewise provides a pharmaceutical composition comprising a peptide or protein drug and an excipient with a pKₐ value of 12 or higher for use as a medicament, wherein said pharmaceutical composition is to be administered transmucosally. The invention also relates to a pharmaceutical composition comprising a peptide or protein drug and an excipient with a pKₐ value of 12 or higher for use in treating or preventing a disease/disorder, wherein said pharmaceutical composition is to be administered transmucosally.

The invention further refers to the use of a peptide or protein drug and an excipient with a pKₐ value of 12 or higher in the preparation of a pharmaceutical composition for transmucosal administration.

Moreover, the invention provides a method of treating or preventing a disease/disorder, the method comprising transmucosally administering, to a subject (e.g., a human) in need thereof, a pharmaceutical composition comprising a peptide or protein drug and an excipient with a pKₐ value of 12 or higher. It will be understood that the disease/disorder to be treated or prevented is a disease/disorder susceptible to treatment or prevention with said peptide or protein drug.

The invention likewise relates to a method of transmucosally delivering a peptide or protein drug, the method comprising transmucosally administering a pharmaceutical composition comprising said peptide or protein drug and an excipient with a pKₐ value of 12 or higher to a subject (e.g., a human) in need thereof.

The peptide or protein drug to be administered in accordance with the present invention preferably has a molecular weight of equal to or less than about 300 kDa (such as, e.g., equal to or less than about 260 kDa, or equal to or less than about 220 kDa, or equal to or less than about 180 kDa, or equal to or less than about 150 kDa, or equal to or less than about 120 kDa, or equal to or less than about 100 kDa, or equal to or less than about 90 kDa, or equal to or less than about 80 kDa, or equal to or less than about 70 kDa, or equal to or less than about 60 kDa, or equal to or less than about 50 kDa, or equal to or less than about 40 kDa, or equal to or less than about 30 kDa, or equal to or less than about 20 kDa, or equal to or less than about 10 kDa, or equal to or less than about 5 kDa, or equal to or less than about 2 kDa, or equal to or less than about 1 kDa, or equal to or less than about 500 Da). More preferably, the peptide or protein drug has a maximum molecular weight of equal to or less than about 200 kDa, even more preferably equal to or less than about 150 kDa, even more preferably equal to or less than about 100 kDa, even more preferably equal to or less than about 50 kDa, even more preferably equal to or less than about 40 kDa, even more preferably equal to or less than about 30 kDa, even more preferably equal to or less than about 20 kDa, and yet even more preferably equal to or less than about 10 kDa. It is furthermore preferred that the peptide or protein drug has a minimum molecular weight of equal to or greater than about 300 Da, more preferably equal to or greater than about 500 Da, even more preferably equal to or greater than about 800 Da, and yet even more preferably equal to or greater than about 1 kDa. Accordingly, it is particularly preferred that the peptide or protein drug has a molecular weight of about 300 Da to about 150 kDa, more preferably about 300 Da to about 50 kDa, even more preferably about 500 Da to about 30 kDa, even more preferably about 500 Da to about 20 kDa, and yet even more preferably about 800 Da to about 10 kDa. For oral administration, it is particularly preferred that the peptide or protein drug has a molecular weight of about 1 kDa to about 6 kDa. For nasal administration, a molecular weight of about 1 kDa to about 10 kDa is particularly preferred.

The molecular weight of the peptide or protein drug is indicated herein in dalton (Da), which is an alternative name for the unified atomic mass unit (u). A molecular weight of, e.g., 500 Da is thus equivalent to 500 g/mol. The term “kDa” (kilodalton) refers to 1000 Da.

The molecular weight of the peptide or protein drug can be determined using methods known in the art, such as, e.g., mass spectrometry (e.g., electrospray ionization mass spectrometry (ESI-MS) or matrix-assisted laser desorption/ionization mass spectrometry (MALDI-MS)), gel electrophoresis (e.g., polyacrylamide gel electrophoresis using sodium dodecyl sulfate (SDS-PAGE)), hydrodynamic methods (e.g., gel filtration chromatography or gradient sedimentation), or static light scattering (e.g., multi-angle light scattering (MALS)). It is preferred that the molecular weight of the peptide or protein drug is determined using mass spectrometry.

The peptide or protein drug may be any peptide or protein that is suitable to be used as a medicament. For example, the peptide or protein drug may be a linear peptide or protein drug or a cyclic peptide or protein drug (e.g., a cyclic peptide or protein drug that is cyclized via at least one ester linkage and/or at least one amide linkage; such as, e.g., a cyclotide; cyclotides are disulfide rich peptides characterized by their head-to-tail cyclized peptide backbone and the interlocking arrangement of their disulfide bonds). It may also be a modified or derivatized peptide or protein drug, such as a PEGylated peptide or protein drug or a fatty acid acylated peptide or protein drug or a fatty diacid acylated peptide or protein drug. Moreover, the peptide or protein drug may be free of histidine residues and/or free of cysteine residues. It is generally preferred that the peptide or protein drug is water-soluble, particularly at neutral pH (i.e., at about pH 7). It is furthermore preferred that the peptide or protein drug has at least one serine protease cleavage site, i.e., that the peptide or protein drug comprises one or more amino acid residue(s) amenable or prone to cleavage by a serine protease; more preferably, the peptide or protein drug comprises one or more amino acid residue(s) amenable or prone to cleavage by a serine protease. The term “peptide or protein drug” is used herein synonymously with “therapeutic peptide or protein” and “therapeutic peptide or protein drug”.

The peptide or protein drug is preferably selected from insulin (preferably human insulin), an insulin analog (e.g., a long acting basal insulin analog or a protease stabilized long acting basal insulin analog; exemplary insulin analogs include, without limitation, insulin lispro, insulin PEGlispro, the insulin derivative “A14E, B25H, B29K(N(eps)octadecanedioyl-gGlu-OEG-OEG), desB30 human insulin” (see, e.g., US 2014/0056953 A1), insulin aspart, insulin glulisine, insulin glargine, insulin detemir, NPH insulin, insulin degludec, and the insulin analogs/derivatives described in US 2014/0056953 A1, which is incorporated herein by reference, particularly each one of the insulin analogs/derivatives described in paragraphs [0225] to [0332] of US 2014/0056953 A1), GLP-1, a GLP-1 analog (e.g., an acylated GLP-1 analog or a diacylated GLP-1 analog) or GLP-1 agonist (also referred to as “glucagon-like peptide-1 receptor agonist” or “GLP-1 receptor agonist”), semaglutide, liraglutide, exenatide, exendin-4, lixisenatide, taspoglutide, albiglutide, dulaglutide, langlenatide, beinaglutide, efpeglenatide, GLP-1(7-37), GLP-1(7-36)NH₂, a dual agonist of the GLP-1 receptor and another receptor (e.g., a dual agonist of the GLP-1 receptor and the glucagon receptor, or a dual agonist of the GLP-1 receptor and the gastric inhibitory polypeptide (GIP) receptor), oxyntomodulin, GLP-2, a GLP-2 agonist or analog (e.g., teduglutide or elsiglutide), glucose-dependent insulinotropic polypeptide (also referred to as “gastric inhibitory polypeptide” or GIP), a dual GLP-1 analog, a dual agonist of the glucagon-like peptide 1 receptor and the glucagon receptor (a GLP-1R/GCGR dual agonist), a GLP1/glucagon receptor co-agonist (such as, e.g., any one of the compounds referred to in WO 2015/185640), a dual agonist of the glucagon-like peptide 1 receptor and the gastric inhibitory polypeptide receptor (a GLP-1R/GIPR dual agonist; such as, e.g., any one of the compounds referred to in WO 2013/164483), a GLP1/GIP receptor co-agonist, an exendin-4 peptide analog (particularly an exendin-4 peptide analog which is a GLP-1R/GIPR dual agonist; such as, e.g., any one of the exendin-4 peptide analogs referred to in WO 2015/086728), an exendin-4 derivative (particularly an exendin-4 derivative which is a GLP-1R/GCGR dual agonist; such as, e.g., any one of the exendin-4 derivatives referred to in WO 2015/155139 or in WO 2015/086733), elamipretide, a cyclotide (i.e., a peptide characterized by its head-to-tail cyclised peptide backbone and the interlocking arrangement of its disulfide bonds; including, e.g., a cyclotide having at least two disulfide bonds, and preferably a cyclotide having three disulfide bonds), recombinant factor VIIa (rFVIIa), eptacog alfa, amylin, an amylin analog, pramlintide, a somatostatin analog (e.g., octreotide, lanreotide, or pasireotide), goserelin (e.g., goserelin acetate), buserelin (e.g., buserelin acetate), leptin, a leptin analog (e.g., metreleptin), peptide YY (PYY), a PYY analog, glatiramer (e.g., glatiramer acetate), leuprolide (e.g., leuprolide acetate), desmopressin (e.g., desmopressin acetate, particularly desmopressin monoacetate trihydrate), a desmopressin analog, a vasopressin receptor 2 (V2 receptor) agonist peptide, osteocalcin, an osteocalcin analog or derivative, human growth hormone (hGH), a human growth hormone analog, a long-acting human growth hormone (such as, e.g., somapacitan or hGH-CTP (human growth hormone derivatized with the C-terminal peptide (CTP) of the beta chain of human chorionic gonadotropin (hCG)), fibroblast growth factor 21 (FGF21), an antibody (e.g., any of the exemplary antibodies described herein below), a glycopeptide antibiotic (e.g., a glycosylated cyclic or polycyclic nonribosomal peptide such as vancomycin, teicoplanin, telavancin, bleomycin, ramoplanin, or decaplanin), a cyclotide, bortezomib, cosyntropin, chorionic gonadotropin, menotropin, sermorelin, luteinizing-hormone-releasing hormone (LHRH; also referred to as “gonadotropin-releasing hormone”), somatropin, calcitonin (e.g., calcitonin-salmon), pentagastrin, oxytocin, neseritide, anakinra, enfuvirtide, pegvisomant, dornase alfa, lepirudin, anidulafungin, eptifibatide, interferon alfacon-1, interferon alpha-2a, interferon alpha-2b, interferon beta-1a, interferon beta-1b, interferon gamma-1 b, peginterferon alfa-2a (i.e., pegylated interferon alfa-2a), peginterferon alfa-2b (i.e., pegylated interferon alfa-2b), peginterferon beta-1a (i.e., pegylated interferon beta-1a), fibrinolysin, vasopressin, aldesleukin, an epoetin, epoetin alfa, darbepoetin alfa, epoetin beta, epoetin delta, epoetin omega, epoetin zeta, epoetin theta, methoxy polyethylene glycol-epoetin beta, continuous erythropoietin receptor activator (CERA; a pegylated EPO derivative), peglylated epo, albupoetin, an epo-dimer analogue, epo-Fc, carbamylated EPO (CEPO), synthetic erythropoese protein (SEP), low molecular epo analogue (P131-1402), filgrastim, PEG-filgrastim, interleukin-11, cyclosporine, glucagon, urokinase, viomycin, thyrotropin-releasing hormone (TRH), leucine-enkephalin, methionine-enkephalin, substance P (CAS no. 33507-63-0), adrenocorticotropic hormone (ACTH), parathyroid hormone (PTH), a parathyroid hormone (PTH) fragment (e.g., teriparatide (also referred to as “PTH(1-34)”), PTH(1-31), or PTH(2-34)), parathyroid hormone-related protein (PTHrP), abaloparatide, linaclotide, carfilzomib, icatibant, ecallantide, cilengitide, a prostaglandin F2a receptor modulator (e.g., PDC31), abciximab (C7E3-Fab), ranibizumab, alefacept, romiplostim, anakinra, abatacept, belatacept, and pharmaceutically acceptable salts thereof. If the subject/patient to be treated is a human and if the peptide or protein drug is an endogenous peptide or protein in human beings (i.e., occurs naturally in humans; such as, e.g., insulin or glucagon), it is furthermore preferred to use a human isoform of the corresponding peptide or protein (which may, e.g., be recombinantly expressed or chemically synthesized).

More preferably, the peptide or protein drug is selected from GLP-1, a GLP-1 analog (e.g., an acylated GLP-1 analog or a diacylated GLP-1 analog, or a long-acting albumin-binding fatty acid-derivatized GLP-1 analog), a GLP-1 agonist, semaglutide, liraglutide, exenatide, exendin-4, lixisenatide, taspoglutide, albiglutide, dulaglutide, langlenatide, beinaglutide, efpeglenatide, GLP-1(7-37), GLP-1(7-36)NH₂, a dual agonist of the GLP-1 receptor and another receptor (e.g., a dual agonist of the GLP-1 receptor and the glucagon receptor, or a dual agonist of the GLP-1 receptor and the GIP receptor), oxyntomodulin, GLP-2, a GLP-2 agonist or analog (e.g., teduglutide or elsiglutide), recombinant factor Vila (rFVIIa), eptacog alfa, amylin, an amylin analog, pramlintide, a somatostatin analog (e.g., octreotide, lanreotide, or pasireotide), goserelin (e.g., goserelin acetate), buserelin, peptide YY (PYY), a PYY analog, glatiramer (e.g., glatiramer acetate), leuprolide (e.g., leuprolide acetate), desmopressin (e.g., desmopressin acetate, particularly desmopressin monoacetate trihydrate), a desmopressin analog, a vasopressin receptor 2 (V2 receptor) agonist peptide, teicoplanin, telavancin, bleomycin, ramoplanin, decaplanin, bortezomib, cosyntropin, sermorelin, luteinizing-hormone-releasing hormone (LHRH), calcitonin (e.g., calcitonin-salmon), pentagastrin, neseritide, enfuvirtide, eptifibatide, cyclosporine, glucagon, viomycin, thyrotropin-releasing hormone (TRH), leucine-enkephalin, methionine-enkephalin, substance P, a parathyroid hormone (PTH) fragment (e.g., teriparatide (PTH(1-34)), PTH(1-31), or PTH(2-34)), carfilzomib, icatibant, cilengitide, a prostaglandin F2a receptor modulator (e.g., PDC31), and pharmaceutically acceptable salts thereof.

Even more preferably, the peptide or protein drug is selected from a GLP-1 agonist, semaglutide, liraglutide, exenatide, exendin-4, lixisenatide, taspoglutide, albiglutide, dulaglutide, langlenatide, beinaglutide, efpeglenatide, GLP-1(7-37), GLP-1(7-36)NH₂, a dual agonist of the GLP-1 receptor and another receptor (e.g., a dual agonist of the GLP-1 receptor and the glucagon receptor, or a dual agonist of the GLP-1 receptor and the GIP receptor), oxyntomodulin, GLP-2, a GLP-2 agonist or analog (e.g., teduglutide or elsiglutide), a somatostatin analog (e.g., octreotide, lanreotide, or pasireotide), desmopressin (e.g., desmopressin acetate, particularly desmopressin monoacetate trihydrate), a desmopressin analog, a vasopressin receptor 2 (V2 receptor) agonist peptide, a parathyroid hormone (PTH) fragment (e.g., teriparatide (PTH(1-34)), PTH(1-31), or PTH(2-34)), and pharmaceutically acceptable salts thereof. For example, the peptide or protein drug may be a GLP-1 agonist (such as liraglutide), a PTH fragment (such as teriparatide, i.e. PTH(1-34)), a somatostatin analog (such as octreotide), or desmopressin (e.g., desmopressin acetate, particularly desmopressin monoacetate trihydrate).

Yet even more preferably, the peptide or protein drug is selected from a GLP-1 agonist, semaglutide, liraglutide, exenatide, exendin-4, lixisenatide, taspoglutide, albiglutide, dulaglutide, langlenatide, beinaglutide, efpeglenatide, GLP-1(7-37), GLP-1(7-36)NH₂, a dual agonist of the GLP-1 receptor and the glucagon receptor, oxyntomodulin, and pharmaceutically acceptable salts thereof.

As noted above, the peptide or protein drug may be an insulin analog. The insulin analog is preferably selected from:
B29K(N(ε)hexadecanedioyl-γ-L-Glu) A14E B25H desB30 human insulin;
B29K(N(ε)octadecanedioyl-γ-L-Glu-OEG-OEG) desB30 human insulin;
B29K(N(ε)octadecanedioyl-γ-L-Glu) A14E B25H desB30 human insulin;
B29K(N(ε)eicosanedioyl-γ-L-Glu) A14E B25H desB30 human insulin;
B29K(N(ε)octadecanedioyl-γ-L-Glu-OEG-OEG) A14E B25H desB30 human insulin;
B29K(N(ε)eicosanedioyl-γ-L-Glu-OEG-OEG) A14E B25H desB30 human insulin;
B29K(N(ε)eicosanedioyl-γ-L-Glu-OEG-OEG) A14E B16H B25H desB30 human insulin;
B29K(N(ε)hexadecanedioyl-γ-L-Glu) A14E B16H B25H desB30 human insulin;
B29K(N(ε)eicosanedioyl-γ-L-Glu-OEG-OEG) A14E B16H B25H desB30 human insulin; and
B29K(N(ε)octadecanedioyl) A14E B25H desB30 human insulin.

These insulin analogs are described and characterized, e.g., in US 2014/0056953 A1. It is particularly preferred that the insulin analog is B29K(N(ε)octadecanedioyl-γ-L-Glu-OEG-OEG) A14E B25H desB30 human insulin.

Moreover, as also described above, the peptide or protein drug may be a GLP-1 analog. The GLP-1 analog may be, in particular, a variant of the human Glucagon-Like Peptide-1, preferably a variant of GLP-1(7-37). The amino acid sequence of GLP-1(7-37) is HAEGTFTSDVSSYLEGQAAKEFIAWLVKGRG. The aforementioned “variant” of human Glucagon-Like Peptide-1 or of GLP-1(7-37) preferably refers to a compound differing by one or more amino acids from human Glucagon-Like Peptide-1 or from GLP-1(7-37), respectively, wherein such difference is caused by the addition, substitution or deletion of at least one amino acid (e.g., 1 to 10 amino acids) or any combination of such addition(s), substitution(s) and/or deletion(s). A GLP-1 analog may, e.g., exhibit at least 60% (preferably at least 65%, more preferably at least 70%, even more preferably at least 80%, and most preferably at least 90%) sequence identity to GLP-1(7-37) over the entire length of said GLP-1(7-37). As an example of a method for the determination of sequence identity between a GLP-1 analog and GLP-1(7-37), the two peptides [Aib8]GLP-1(7-37) and GLP-1(7-37) are aligned. [Aib8]GLP-1(7-37) differs from GLP-1(7-37) in that the alanine in position 8 is replaced by α-methylalanine (Aib, i.e. 2-aminoisobutyric acid). The sequence identity of [Aib8]GLP-1(7-37) relative to GLP-1(7-37) is given by the number of aligned identical residues minus the number of different residues divided by the total number of residues in GLP-1(7-37). Accordingly, in this example the sequence identity is (31-1)/31. The C-terminus of the GLP-1 analog (including any one of the specific GLP-1 analogs described herein) may also be in the form of an amide. Moreover, the GLP-1 analog may be, e.g., GLP-1(7-37) or GLP-1(7-36)amide. The GLP-1 analog may also be, e.g., exendin-4, the amino acid sequence of which is HGEGTFITSDLSKQMEEEAVRLFIEWLKNGGPSSGAPPPS. The GLP-1 analog may further be a modified form of naturally occurring GLP-1 (particularly human GLP-1), which differs from the GLP-1 peptide in that it comprises one substituent which is covalently attached to the peptide. Said substituent may comprise a fatty acid (e.g., a C16, C18 or C20 fatty acid) or a fatty diacid (e.g., a C16, C18 or C20 fatty diacid). Said substituent may also comprise a group of the following formula:

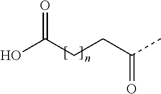

wherein n is at least 13 (e.g., 13, 14, 15, 16, 17, 18 or 19; preferably 13 to 17; more preferably 13, 15 or 17). Said substituent may also comprise one or more 8-amino-3,6-dioxaoctanoic acid (OEG) groups, such as two OEG groups. In particular, said substituent may be selected from [2-(2-{2-[2-(2-{2-[(S)-4-carboxy-4-(17-carboxyheptadecanoylamino)butyrylamino]ethoxy}ethoxy)acetylamino]ethoxy}ethoxy)acetyl] and [2-(2-{2-[2-(2-{2-[(S)-4-carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl]. The GLP-1 analog may also be selected from one or more of the GLP-1 agonists disclosed in WO 93/19175, WO 96/29342, WO 98/08871, WO 99/43707, WO 99/43706, WO 99/43341, WO 99/43708, WO 2005/027978, WO 2005/058954, WO 2005/058958, WO 2006/005667, WO 2006/037810, WO 2006/037811, WO 2006/097537, WO 2006/097538, WO 2008/023050, WO 2009/030738, WO 2009/030771 and WO 2009/030774.

The peptide or protein drug may also be an antibody, preferably a monoclonal antibody, and it may in particular be a single-chain antibody or a single-domain antibody (e.g., a “nanobody”). Such therapeutic antibodies are preferably administered via the nasal route. An example of a nanobody, which can be used as the peptide or protein drug in accordance with the invention, is caplacizumab. Caplacizumab is a single-domain antibody which can be used, e.g., in the treatment or prevention of thrombotic thrombocytopenic purpura or of thrombosis.

In particular, the peptide or protein drug may be an antibody selected from 3F8, 8H9, abagovomab, abciximab, abituzumab, abrezekimab, abrilumab, actoxumab, adalimumab, adecatumumab, atidortoxumab, aducanumab, afasevikumab, afelimomab, afutuzumab, alacizumab pegol, alemtuzumab, alirocumab, altumomab pentetate, amatuximab, anatumomab mafenatox, andecaliximab, anetumab ravtansine, anifrolumab, anrukinzumab, apolizumab, aprutumab ixadotin, arcitumomab, ascrinvacumab, aselizumab, atezolizumab, atinumab, atorolimumab, avelumab, azintuxizumab vedotin, bapineuzumab, basiliximab, bavituximab, BCD-100, bectumomab, begelomab, belantamab mafodotin, belimumab, bemarituzumab, benralizumab, berlimatoxumab, bersanlimab, bertilimumab, besilesomab, bevacizumab, bezlotoxumab, biciromab, bimagrumab, bimekizumab, birtamimab, bivatuzumab mertansine, BIVV009, bleselumab, blinatumomab, blontuvetmab, blosozumab, bococizumab, brazikumab, brentuximab vedotin, briakinumab, brodalumab, brolucizumab, brontictuzumab, burosumab, cabiralizumab, camidanlumab tesirine, camrelizumab, canakinumab, cantuzumab mertansine, cantuzumab ravtansine, caplacizumab, capromab pendetide, carlumab, carotuximab, catumaxomab, cbr96-doxorubicin immunoconjugate, cedelizumab, cemiplimab, cergutuzumab amunaleukin, certolizumab pegol, cetrelimab, cetuximab, cibisatamab, citatuzumab bogatox, cixutumumab, clazakizumab, clenoliximab, clivatuzumab tetraxetan, codrituzumab, cofetuzumab pelidotin, coltuximab ravtansine, conatumumab, concizumab, cosfroviximab, crenezumab, crizanlizumab, crotedumab, CR6261, cusatuzumab, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, detumomab, dezamizumab, dinutuximab, diridavumab, domagrozumab, dorlimomab aritox, drozitumab, DS-8201, duligotuzumab, dupilumab, durvalumab, dusigitumab, duvortuxizumab, ecromeximab, eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, enapotamab edotin, enavatuzumab, enfortumab vedotin, enlimomab pegol, enoblituzumab, enokizumab, enoticumab, ensituximab, epitumomab cituxetan, epratuzumab, eptinezumab, erenumab, erlizumab, ertumaxomab, etaracizumab, etigilimab, etrolizumab, evinacumab, evolocumab, exbivirumab, fanolesomab, faralimomab, faricimab, farletuzumab, fasinumab, FBTA05, felvizumab, fezakinumab, fibatuzumab, ficlatuzumab, figitumumab, firivumab, flanvotumab, fletikumab, flotetuzumab, fontolizumab, foralumab, foravirumab, fremanezumab, fresolimumab, frunevetmab, fulranumab, futuximab, galcanezumab, galiximab, gancotamab, ganitumab, gantenerumab, gatipotuzumab, gavilimomab, gedivumab, gemtuzumab ozogamicin, gevokizumab, gilvetmab, gimsilumab, girentuximab, glembatumumab vedotin, golimumab, gomiliximab, gosuranemab, guselkumab, ianalumab, ibalizumab, IBI308, ibritumomab tiuxetan, icrucumab, idarucizumab, ifabotuzumab, igovomab, iladatuzumab vedotin, IMAB362, imalumab, imaprelimab, imciromab, imgatuzumab, inclacumab, indatuximab ravtansine, indusatumab vedotin, inebilizumab, infliximab, intetumumab, inolimomab, inotuzumab ozogamicin, ipilimumab, iomab-b, iratumumab, isatuximab, iscalimab, istiratumab, itolizumab, ixekizumab, keliximab, labetuzumab, lacnotuzumab, ladiratuzurnab vedotin, lampalizumab, lanadelumab, landogrozumab, laprituximab emtansine, larcaviximab, lebrikizumab, lemalesomab, lendalizumab, lenvervimab, lenzilumab, lerdelimumab, ieronlimab, lesofavumab, letolizumab, lexatumumab, libivirumab, lifastuzumab vedotin, ligelizumab, loncastuximab tesirine, losatuxizumab vedotin, lilotomab satetraxetan, lintuzumab, lirilumab, lodelcizumab, lokivetmab, lorvotuzumab mertansine, lucatumumab, lulizumab pegol, lumiliximab, lumretuzumab, lupartumab amadotin, lutikizumab, MABp1, mapatumumab, margetuximab, marstacimab, maslimomab, mavrilimumab, matuzumab, mepolizumab, metelimumab, milatuzumab, minretumomab, mirikizumab, mirvetuximab soravtansine, mitumomab, modotuximab, mogamulizumab, monalizumab, morolimumab, mosunetuzumab, motavizumab, moxetumomab pasudotox, muromonab-CD3, nacolomab tafenatox, namiumab, naptumomab estafenatox, naratuximab emtansine, narnatumab, natalizumab, navicixizumab, navivumab, naxitamab, nebacumab, necitumumab, nemolizumab, NEOD001, nerelimomab, nesvacumab, netakimab, nimotuzumab, nirsevimab, nivolumab, nofetumomab merpentan, obiltoxaximab, obinutuzumab, ocaratuzumab, ocrelizumab, odulimomab, ofatumumab, olaratumab, oleclumab, olendalizumab, olokizumab, omalizumab, OMS721, onartuzumab, ontuxizumab, onvatilimab, opicinumab, oportuzumab monatox, oregovomab, orticumab, otelixizumab, otilimab, otlertuzumab, oxelumab, ozanezumab, ozoralizumab, pagibaximab, paiivizumab, pamrevlumab, panitumumab, pankomab, panobacumab, parsatuzumab, pascolizumab, pasotuxizumab, pateclizumab, patritumab, PDR001, pembrolizumab, pemtumomab, perakizumab, pertuzumab, pexelizumab, pidilizumab, pinatuzumab vedotin, pintumomab, placulumab, plozalizumab, pogalizumab, polatuzumab vedotin, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, anibizumab, raxibacumab, ravagalimab, ravulizumab, refanezumab, regavirumab, remtolumab, reslizumab, rilotumumab, rinucumab, risankizumab, rituximab, rivabazumab pegol, robatumumab, rmab, roledumab, romilkimab, romosozumab, rontalizumab, rosmantuzumab, rovalpituzumab tesirine, rovelizumab, rozanolixizumab, ruplizumab, SA237, sacituzumab govitecan, samalizumab, samrotamab vedotin, sapelizumab, sarilumab, satralizumab, satumomab pendetide, secukinumab, selicrelumab, seribantumab, setoxaximab, setrusumab, sevirumab, sibrotuzumab, SGN-CD19A, SHP647, sifalimumab, siltuximab, simtuzumab, siplizumab, sirtratumab vedotin, sirukumab, sofituzumab vedotin, solanezumab, solitomab, sonepcizumab, sontuzumab, spartalizumab, stamulumab, sulesomab, suptavumab, sutimlimab, suvizumab, suvratoxumab, tabalumab, tacatuzumab etraxetan, tadocizumab, talacotuzumab, talizumab, tamtuvetmab, tanezumab, taplitumomab paptox, tarextumab, tavolimab, tefibazumab, telimomab aritox, telisotuzumab vedotin, tenatumomab, teneliximab, teplizumab, tepoditamab, teprotumumab, tesidolumab, tetulomab, tezepelumab, TGN1412, tibulizumab, tildrakizumab, tigatuzumab, timigutuzumab, timolumab, tiragotumab, tislelizumab, tisotumab vedotin, TNX-650, tocilizumab, tomuzotuximab, toralizumab, tosatoxumab, tositumomab, tovetumab, tralokinumab, trastuzumab, trastuzumab emtansine, TRBS07, tregalizumab, tremelimumab, trevogrumab, tucotuzumab celmoleukin, tuvirumab, ublituximab, ulocuplumab, urelumab, urtoxazumab, ustekinumab, utomilumab, vadastuximab talirine, vanalimab, vandortuzumab vedotin, vantictumab, vanucizumab, vapaliximab, varisacumab, varlilumab, vatelizumab, vedolizumab, veltuzumab, vepalimomab, vesencumab, visilizumab, vobarilizumab, volociximab, vonlerolizumab, vopratelimab, vorsetuzumab mafodotin, votumumab, vunakizumab, xentuzumab, XMAB-5574, zalutumumab, zanolimumab, zatuximab, zenocutuzumab, ziralimumab, zolbetuximab, and zolimomab aritox.

The peptide or protein drug to be used in accordance with the invention can also be a mixture of two or more different peptide or protein drugs, including any of the above-mentioned specific peptide or protein drugs. For example, the peptide or protein drug may be a mixture of human insulin and a GLP-1 agonist (e.g. semaglutide, liraglutide, exenatide, exendin-4, lixisenatide, taspoglutide, albiglutide, dulaglutide, langlenatide, beinaglutide, or efpeglenatide).

The above-described exemplary peptide or protein drugs have been proposed in the literature to be suitable for the treatment or prevention of various different diseases/disorders, and some of these peptide or protein drugs have already received marketing authorizations for specific therapeutic indications. The present invention also specifically relates to the pharmaceutical composition provided herein for use in the treatment or prevention of a disease/disorder that is amenable to be treated or prevented with the respective peptide or protein drug. Likewise, the invention relates to a method of treating or preventing a disease/disorder, the method comprising transmucosally administering, to a subject in need thereof, a pharmaceutical composition comprising a peptide or protein drug and an excipient with a pKₐ value of 12 or higher, wherein said disease/disorder is a disease/disorder that is amenable to be treated or prevented with the respective peptide or protein drug. Preferred examples of diseases/disorders that are amenable to be treated or prevented with any particular peptide or protein drug in accordance with the present invention are disclosed in (and can be derived from) the medical literature, particularly from any one of: ROTE LISTE online edition (in the version as of the priority date or the filing date of the present specification); ROTE LISTE 2017 (print edition, Rote Liste Service GmbH, 2017, ISBN 978-3946057109); GELBE LISTE online edition (in the version as of the priority date or the filing date of the present specification); GELBE LISTE 2017 (print edition, AVOXA—Mediengruppe Deutscher Apotheker GmbH, 2017, ISBN 978-3774199149); Aktories K et al. (eds.), Allgemeine und spezielle Pharmakologie und Toxikologie, Urban & Fischer Verlag/Elsevier GmbH, 12^{th} edition, 2017, ISBN 978-3437425257; Ammon HPT et al. (eds.), Hunnius Pharmazeutisches Worterbuch, De Gruyter, 11^{th} edition, 2014, ISBN 978-3110309904; Otto H H et al., Arzneimittel—Ein Handbuch für Ärzte und Apotheker, Wissenschaftliche Verlagsgesellschaft, 2017, ISBN 978-3804737266; DrugBank (www.drugbank.ca, in the version as of the priority date or the filing date of the present specification); Drugs.com (www.drugs.com, in the version as of the priority date or the filing date of the present specification); Merck Manuals (www.merckmanuals.com, in the version as of the priority date or the filing date of the present specification); European Medicines Agency (EMA) database (www.ema.europa.eu, in the version as of the priority date or the filing date of the present specification); U.S. Food & Drug Administration (U.S. FDA) database (www.fda.gov, in the version as of the priority date or the filing date of the present specification); Japanese Pharmaceuticals and Medical Devices Agency database (www.pmda.go.jp, in the version as of the priority date or the filing date of the present specification); or electronic Medicines Compendium (eMC) database (www.medicines.org.uk/emc/, in the version as of the priority date or the filing date of the present specification). Examples of diseases/disorders that are amenable to be treated or prevented with an analog or derivative of any particular peptide or protein drug include the same diseases/disorders that are amenable to be treated or prevented with the corresponding (underivatized) peptide or protein drug. Moreover, preferred examples of diseases/disorders that are amenable to be treated or prevented with any of the above-mentioned insulin or insulin analogs include, in particular, diabetes (e.g., type 1 diabetes mellitus or type 2 diabetes mellitus); preferred examples of diseases/disorders that are amenable to be treated or prevented with any of the above-mentioned GLP-1 peptides or GLP-1 receptor agonists include, in particular, diabetes, obesity, or non-alcoholic fatty liver disease (NASH); preferred examples of diseases/disorders that are amenable to be treated or prevented with buserelin include, in particular, hormone-responsive cancer (such as, e.g., prostate cancer or breast cancer), or estrogen-dependent conditions (such as, e.g., endometriosis or uterine fibroids); buserelin can further be used, e.g., in assisted reproduction; preferred examples of diseases/disorders that are amenable to be treated or prevented with human growth hormone (hGH) or any of the above-mentioned hGH analogs or derivatives include, in particular, growth hormone deficiency; preferred examples of diseases/disorders that are amenable to be treated or prevented with fibroblast growth factor 21 (FGF21) include, in particular, cardiovascular disease, obesity, or diabetes (particularly type 2 diabetes); preferred examples of diseases/disorders that are amenable to be treated or prevented with any of the above-mentioned epoetin or any of its analogues or derivatives include, in particular, anemia, Alzheimer's disease (see, e.g., Maurice T et al., J Psychopharmacol. 2013; 27(11): 1044-57), Parkinson's disease (see, e.g., Alcalá-Barraza S R et al., J Drug Target. 2010; 18(3): 179-90), or multiple sclerosis; preferred examples of diseases/disorders that are amenable to be treated or prevented with the above-mentioned filgrastim or any derivatives thereof (e.g., PEG-filgrastim) include, in particular, low blood neutrophils due to a number of causes such as, e.g., chemotherapy, radiation poisoning, HIV or AIDS, or unknown causes; preferred examples of diseases/disorders that are amenable to be treated or prevented with the above-mentioned antibody adalimumab include, in particular, inflammatory or autoimmune diseases/disorders, and are more preferably selected from rheumatoid arthritis, psoriatic arthritis, ankylosing spondylitis, Crohn's disease, ulcerative colitis, psoriasis (e.g., chronic psoriasis), hidradenitis suppurativa, juvenile idiopathic arthritis, and birdshot retinochoroidopathy; preferred examples of diseases/disorders that are amenable to be treated or prevented with the above-mentioned antibody ustekinumab include, in particular, inflammatory or autoimmune diseases/disorders (e.g., intestinal inflammatory or intestinal autoimmune diseases/disorders), and are more preferably selected from Crohn's disease, ulcerative colitis, psoriasis (e.g., chronic psoriasis), and psoriatic arthritis.

The pharmaceutical composition according to the present invention can also be used to treat or prevent an intestinal disease/disorder, particularly an inflammatory, infectious or cancerous intestinal disease/disorder, such as, e.g., inflammatory bowel disease, Crohn's disease, ulcerative colitis, irritable bowel syndrome, a colonic bacterial infectious disease, or colorectal cancer. It will be understood that the peptide or protein drug comprised in the pharmaceutical composition should in this case be a peptide or protein drug (particularly an antibody; e.g., adalimumab) effective against the respective intestinal disease/disorder. The corresponding pharmaceutical composition is preferably administered orally, and is preferably formulated so as to release the peptide or protein drug in the ileum and/or in the colon. In particular, the pharmaceutical composition may be provided as a dosage form (e.g., a capsule, multiparticulate or tablet) having an enteric coating.

The excipient with a pKₐ value of 12 or higher, which is to be used in accordance with the present invention, is not particularly limited. The excipient has a pKₐ value of 12 or higher, such as, e.g., a pKₐ of 12 to 14, or a pKₐ of 12 to 13. The pKₐ value of an excipient can be determined, e.g., by potentiometric titration, calorimetry (e.g., isothermal titration calorimetry), UV/VIS spectrophotometry, conductometry, or nuclear magnetic resonance (NMR). In particular, the pKₐ value can be determined by the complementary use of potentiometry and NMR spectroscopy (e.g., as described in Fitch C A et al., Protein Sci. 2015; 24(5):752-61). It will further be understood that the excipient is pharmaceutically acceptable.

Preferably, the excipient with a pKₐ value of 12 or higher is arginine free base (i.e., 2-amino-5-guanidinopentanoic acid in free base form; CAS 7200-25-1), EDTA tetrasodium salt (i.e., tetrasodium ethylenediaminetetraacetate; including in particular, anhydrous tetrasodium EDTA, or tetrasodium EDTA hydrate), trisodium phosphate (i.e., Na₃PO₄; including, in particular, anhydrous trisodium phosphate (CAS 7601-54-9), partially hydrated trisodium phosphate (Na₃PO₄.x H₂O, wherein x is 1, 2, 3, 4, 5, 6, 7, 8, 9, 10 or 11), or fully hydrated trisodium phosphate (Na₃PO₄.12H₂O; CAS 10101-89-0)), tris(hydroxymethyl)aminomethane (“Tris”, i.e. trometamol), lysine (e.g., L-lysine or D-lysine), or calcium hydroxide (i.e., Ca(OH)₂). Arginine free base, trisodium phosphate, lysine, and calcium hydroxide are listed by the U.S. FDA as inactive ingredients in approved drug products, and are therefore particularly suitable for pharmaceutical use. More preferably, the excipient with a pKₐ value of 12 or higher is arginine free base, EDTA tetrasodium salt, or trisodium phosphate. Even more preferably, the excipient with a pKₐ value of 12 or higher is arginine free base or trisodium phosphate. Yet even more preferably, the excipient is arginine free base, particularly L-arginine free base (CAS 74-79-3).

In another embodiment, arginine hydrochloride (particularly L-arginine HCl) may be used in place of the excipient with a pKₐ value of 12 or higher. The present invention thus also relates to a pharmaceutical composition for transmucosal administration, comprising a peptide or protein drug and arginine hydrochloride, and corresponding methods and uses of such a composition, as described herein for the pharmaceutical composition of the present invention (which comprises a peptide or protein drug and an excipient with a pKₐ value of 12 or higher). In this context, the invention particularly relates to a pharmaceutical composition for nasal administration, comprising a peptide or protein drug and arginine hydrochloride, as well as corresponding methods and uses of such a composition.

The pharmaceutical composition according to the present invention (particularly the pharmaceutical composition for transmucosal administration, comprising a peptide or protein drug and an excipient with a pKₐ value of 12 or higher) may also contain arginine hydrochloride (particularly L-arginine HCl) in addition to the excipient with a pKₐ value of 12 or higher. For example, the pharmaceutical composition may contain arginine free base and arginine hydrochloride (particularly L-arginine free base and L-arginine HCl).

The pharmaceutical composition according to the present invention (particularly the pharmaceutical composition for transmucosal administration, comprising a peptide or protein drug and an excipient with a pKₐ value of 12 or higher) may also contain tyrosine (L- or D-tyrosine) in addition to the excipient with a pKₐ value of 12 or higher. For example, the pharmaceutical composition may contain tyrosine and L-arginine free base.

It is particularly preferred that the pharmaceutical composition according to the present invention (particularly the pharmaceutical composition for transmucosal administration, comprising a peptide or protein drug and an excipient with a pKₐ value of 12 or higher) further comprises a permeation enhancer (also referred to as an “absorption enhancer” or “mucosal absorption enhancer”). The administration of a permeation enhancer improves or facilitates the mucosal absorption/permeation of the peptide or protein drug and is advantageous particularly if the peptide or protein drug is a large molecule, e.g., a peptide or protein drug having a molecular weight of about 1 kDa or more.

The permeation enhancer may be, e.g., a zwitter-ionic permeation enhancer, a cationic permeation enhancer, an anionic permeation enhancer (e.g., an anionic permeation enhancer comprising one or more sulfonic acid groups (—SO₃H)), or a non-ionic permeation enhancer. If the excipient with a pKₐ value of 12 or higher is arginine free base (including, e.g., L-arginine free base), it is preferable to use an anionic permeation enhancer (e.g., any one or more of the specific anionic permeation enhancers described herein).

It is preferred that the permeation enhancer is selected from C₈₋₂₀ alkanoyl carnitine (preferably lauroyl carnitine, myristoyl carnitine or palmitoyl carnitine; e.g., lauroyl carnitine chloride, myristoyl carnitine chloride or palmitoyl carnitine chloride), salicylic acid (preferably a salicylate, e.g., sodium salicylate), a salicylic acid derivative (such as, e.g., 3-methoxysalicylic acid, 5-methoxysalicylic acid, or homovanillic acid, a C₈₋₂₀ alkanoic acid (preferably a C₈₋₂₀ alkanoate, more preferably a caprate, a caprylate, a myristate, a palmitate, or a stearate, such as, e.g., sodium caprate, sodium caprylate, sodium myristate, sodium palmitate, or sodium stearate), citric acid (preferably a citrate, e.g., sodium citrate), tartaric acid (preferably a tartrate), a fatty acid acylated amino acid (e.g., any of the fatty acid acylated amino acids described in US 2014/0056953 A1 which is incorporated herein by reference, including, without being limited thereto, sodium lauroyl alaninate, N-dodecanoyl-L-alanine, sodium lauroyl asparaginate, N-dodecanoyl-L-asparagine, sodium lauroyl aspartic acid, N-dodecanoyl-L-aspartic acid, sodium lauroyl cysteinate, N-dodecanoyl-L-cysteine, sodium lauroyl glutamic acid, N-dodecanoyl-L-glutamic acid, sodium lauroyl glutaminate, N-dodecanoyl-L-glutamine, sodium lauroyl glycinate, N-dodecanoyl-L-glycine, sodium lauroyl histidinate, N-dodecanoyl-L-histidine, sodium lauroyl isoleucinate, N-dodecanoyl-L-isoleucine, sodium lauroyl leucinate, N-dodecanoyl-L-leucine, sodium lauroyl methioninate, N-dodecanoyl-L-methionine, sodium lauroyl phenylalaninate, N-dodecanoyl-L-phenylalanine, sodium lauroyl prolinate, N-dodecanoyl-L-proline, sodium lauroyl serinate, N-dodecanoyl-L-serine, sodium lauroyl threoninate, N-dodecanoyl-L-threonine, sodium lauroyl tryptophanate, N-dodecanoyl-L-tryptophane, sodium lauroyl tyrosinate, N-dodecanoyl-L-tyrosine, sodium lauroyl valinate, N-dodecanoyl-L-valine, sodium lauroyl sarcosinate, N-dodecanoyl-L-sarcosine, sodium capric alaninate, N-decanoyl-L-alanine, sodium capric asparaginate, N-decanoyl-L-asparagine, sodium capric aspartic acid, N-decanoyl-L-aspartic acid, sodium capric cysteinate, N-decanoyl-L-cysteine, sodium capric glutamic acid, N-decanoyl-L-glutamic acid, sodium capric glutaminate, N-decanoyl-L-glutamine, sodium capric glycinate, N-decanoyl-L-glycine, sodium capric histidinate, N-decanoyl-L-histidine, sodium capric isoleucinate, N-decanoyl-L-isoleucine, sodium capric leucinate, N-decanoyl-L-leucine, sodium capric methioninate, N-decanoyl-L-methionine, sodium capric phenylalaninate, N-decanoyl-L-phenylalanine, sodium capric prolinate, N-decanoyl-L-proline, sodium capric serinate, N-decanoyl-L-serine, sodium capric threoninate, N-decanoyl-L-threonine, sodium capric tryptophanate, N-decanoyl-L-tryptophane, sodium capric tyrosinate, N-decanoyl-L-tyrosine, sodium capric valinate, N-decanoyl-L-valine, sodium capric sarcosinate, N-decanoyl-L-sarcosine, sodium oleoyl sarcosinate, sodium N-decylleucine, sodium stearoyl glutamate (e.g., Amisoft HS-11 P), sodium myristoyl glutamate (e.g., Amisoft MS-11), sodium lauroyl glutamate (e.g., Amisoft LS-11), sodium cocoyl glutamate (e.g., Amisoft CS-11), sodium cocoyl glycinate (e.g., Amilite GCS-11), sodium N-decyl leucine, sodium cocoyl glycine, sodium cocoyl glutamate, sodium lauroyl alaninate, N-dodecanoyl-L-alanine, sodium lauroyl asparaginate, N-dodecanoyl-L-asparagine, sodium lauroyl aspartic acid, N-dodecanoyl-L-aspartic acid, sodium lauroyl cysteinate, N-dodecanoyl-L-cysteine, sodium lauroyl glutamic acid, N-dodecanoyl-L-glutamic acid, sodium lauroyl glutaminate, N-dodecanoyl-L-glutamine, sodium lauroyl glycinate, N-dodecanoyl-L-glycine, sodium lauroyl histidinate, N-dodecanoyl-L-histidine, sodium lauroyl isoleucinate, N-dodecanoyl-L-isoleucine, sodium lauroyl leucinate, N-dodecanoyl-L-leucine, sodium lauroyl methinoninate, N-dodecanoyl-L-methionine, sodium lauroyl phenylalaninate, N-dodecanoyl-L-phenylalanine, sodium lauroyl prolinate, N-dodecanoyl-L-proline, sodium lauroyl serinate, N-dodecanoyl-L-serine, sodium lauroyl threoninate, N-dodecanoyl-L-threonine, sodium lauroyl tryptophanate, N-dodecanoyl-L-tryptophane, sodium lauroyl tyrosinate, N-dodecanoyl-L-tyrosine, sodium lauroyl valinate, N-dodecanoyl-L-valine, N-dodecanoyl-L-sarcosine, sodium capric alaninate, N-decanoyl-L-alanine, sodium capric asparaginate, N-decanoyl-L-asparagine, sodium capric aspartic acid, N-decanoyl-L-aspartic acid, Sodium capric cysteinate, N-decanoyl-L-cysteine, sodium capric glutamic acid, N-decanoyl-L-glutamic acid, sodium capric glutaminate, N-decanoyl-L-glutamine, sodium capric glycinate, N-decanoyl-L-glycine, sodium capric histidinate, N-decanoyl-L-histidine, sodium capric isoleucinate, N-decanoyl-L-isoleucine, sodium capric leucinate, N-decanoyl-L-leucine, leucine, sodium capric methioninate, N-decanoyl-L-methionine, sodium capric phenylalaninate, N-decanoyl-L-phenylalanine, sodium capric prolinate, N-decanoyl-L-proline, sodium capric serinate, N-decanoyl-L-serine, sodium capric threoninate, N-decanoyl-L-threonine, sodium capric tryptophanate, N-decanoyl-L-tryptophane, sodium capric tyrosinate, N-decanoyl-L-tyrosine, sodium capric valinate, N-decanoyl-L-valine, sodium capric sarcosinate, sodium oleoyl sarcosinate, and pharmaceutically acceptable salts of any of the aforementioned compounds; or, e.g., C₈₋₂₀ alkanoyl sarcosinate (e.g., a lauroyl sarcosinate, such as sodium lauroyl sarcosinate) or one of the 20 standard proteinogenic α-amino acids that is acylated with a C₈₋₂₀ alkanoic acid), an alkylsaccharide (e.g., a C₁₋₂₀ alkylsaccharide, such as, e.g., C₈₋₁₀ alkylpolysaccharide like Multitrope™ 1620-LQ-(MV); or, e.g., n-octyl-beta-D-glucopyranoside, n-dodecyl-beta-D-maltoside, n-tetradecyl-beta-D-maltoside, tridecyl-beta-D-maltoside, sucrose laurate, sucrose stearate, sucrose myristate, sucrose palmitate, sucrose cocoate, sucrose mono-dodecanoate, sucrose mono-tridecanoate, sucrose mono-tetradecanoate, a coco-glucoside, or any of the alkylsaccharides described in U.S. Pat. No. 5,661,130 or in WO 2012/112319 which are herein incorporated by reference), a cyclodextrine (e.g., α-cyclodextrin, β-cyclodextrin, γ-cyclodextrin, methyl-β-cyclodextrin, hydroxypropyl β-cyclodextrin, or sulfobutylether β-cyclodextrin), N-[8-(2-hydroxybenzoyl)amino]caprylic acid (preferably a N-[8-(2-hydroxybenzoyl)amino]caprylate, more preferably sodium N-[8-(2-hydroxybenzoyl)amino]caprylate, also referred to as “SNAG”), a N-[8-(2-hydroxybenzoyl)amino]caprylate derivative (preferably a sodium N-[8-(2-hydroxybenzoyl)amino]caprylate derivative), a thiomer (also referred to as a thiolated polymer; may be synthesized, e.g., by immobilization of sulfhydryl bearing ligands on a polymeric backbone of well-established polymers such as, e.g., polyacrylic acid, carboxymethylcellulose or chitosan; exemplary thiomers include the thiomers that are described in Laffleur F et al., Future Med Chem. 2012, 4(17):2205-16 (doi: 10.4155/fmc.12.165) which is incorporated herein by reference), a mucoadhesive polymer having a vitamin B partial structure (e.g., any of the mucoadhesive polymers described in U.S. Pat. No. 8,980,238 B2 which is incorporated herein by reference; including, in particular, any of the polymeric compounds as defined in any one of claims 1 to 3 of U.S. Pat. No. 8,980,238 B2), a calcium chelating compound (e.g., ethylenediaminetetraacetic acid (EDTA), ethylene glycol tetraacetic acid (EGTA), sodium citrate, or polyacrylic acid), cremophor EL (also referred to as “Kolliphor EL”; CAS no. 61791-12-6), chitosan, N,N,N-trimethyl chitosan, benzalkonium chloride, bestatin, cetylpyridinium chloride, cetyltrimethylammonium bromide, a C₂₋₂₀ alkanol (e.g., ethanol, decanol, lauryl alcohol, myristyl alcohol, or palmityl alcohol), a C₈₋₂₀ alkenol (e.g., oleyl alcohol), a C₈₋₂₀ alkenoic acid (e.g., oleic acid), dextran sulfate, diethyleneglycol monoethyl ether (transcutol), 1-dodecylazacyclo-heptan-2-one (Azone®), caprylocaproyl polyoxylglycerides (such as, e.g., caprylocaproyl polyoxyl-8 glycerides; available, e.g., as Labrasol® or ACCONON® MC8-2), ethyl caprylate, glyceryl monolaurate, lysophosphatidylcholine, menthol, a C₈₋₂₀ alkylamine, a C₈₋₂₀ alkenylamine (e.g., oleylamine), phosphatidylcholine, a poloxamer, polyethylene glycol monolaurate, polyoxyethylene, polypropylene glycol monolaurate, a polysorbate (e.g., polysorbate 20 or polysorbate 80), cholic acid (preferably a cholate, e.g., sodium chlolate), a deoxycholate (e.g., sodium deoxycholate), a chenodeoxycholate (e.g., sodium chenodeoxycholate), sodium glycocholate, sodium glycodeoxycholate, sodium lauryl sulfate (SDS), sodium decyl sulfate, sodium octyl sulfate, sodium laureth sulfate, N-lauryl sarcosinate, decyltrimethyl ammonium bromide, benzyldimethyl dodecyl ammonium chloride, myristyltrimethyl ammonium chloride, dodecyl pyridinium chloride, decyldimethyl ammonio propane sulfonate, myristyldimethyl ammonio propane sulfonate, palmityldimethyl ammonio propane sulfonate, ChemBetaine CAS, ChemBetaine Oleyl, Nonylphenoxypolyoxyethylene, polyoxyethylene sorbitan monolaurate, polyoxyethylene sorbitan monopalmitate, sorbitan monooleate, Triton X-100, hexanoic acid, heptanoic acid, methyl laurate, isopropyl myristate, isopropyl palmitate, methyl palmitate, diethyl sebaccate, sodium oleate, urea, lauryl amine, caprolactam, methyl pyrrolidone, octyl pyrrolidone, methyl piperazine, phenyl piperazine, Carbopol 934P, glyccyrhetinic acid, bromelain, pinene oxide, limonene, cineole, octyl dodecanol, fenchone, menthane, trimethoxy propylene methyl benzene, a cell-penetrating peptide (e.g., KLAKLAK, polyarginine or oligoarginine (particularly octa-arginine), penetratin (particularly L-penetratin), a penetratin analog (particularly PenetraMax; see, e.g., El-Sayed Khafagy et al., Eur J Pharm Biopharm. 2013; 85(3 Pt A):736-43), HIV-1 Tat, transportan, or any of the cell-penetrating peptides referred to in US 2012/0065124), macrogol-15-hydroxystearate (e.g., Solutol HS 15), CriticalSorb (see, e.g., Illum L et al. J Control Release. 2012; 162(1):194-200), a taurocholate (e.g., sodium taurocholate), a taurodeoxycholate (e.g., sodium taurodeoxycholate), a sulfoxide (e.g., a (C₁₋₁₀ alkyl)-(C₁₋₁₀ alkyl)-sulfoxide, such as, e.g., decyl methyl sulfoxide, or dimethyl sulfoxide), cyclopentadecalactone, 8-(N-2-hydroxy-5-chloro-benzoyl)-amino-caprylic acid (also referred to as “5-CNAC”), N-(10-[2-hydroxybenzoyl]amino)decanoic acid (also referred to as “SNAD”), dodecyl-2-N,N-dimethylamino propionate (also referred to as “DDAIP”), D-α-tocopheryl polyethylene glycol-1000 succinate (also referred to as “TPGS”), arginine, and pharmaceutically acceptable salts of the aforementioned compounds. Mixtures of two or more permeation enhancers, including any of the above-described permeation enhancers, can also be used. Moreover, any of the chemical permeation enhancers described in Whitehead K et al. Pharm Res. 2008 June; 25(6):1412-9 (particularly any one of those described in Table I of this reference), any one of the modified amino acids disclosed in U.S. Pat. No. 5,866,536 (particularly any one of compounds I to CXXIII, as disclosed in U.S. Pat. No. 5,866,536 which is incorporated herein by reference, or a pharmaceutically acceptable salt or solvate thereof, such as a disodium salt, an ethanol solvate, or a hydrate of any one of these compounds), any one of the modified amino acids disclosed in U.S. Pat. No. 5,773,647 (particularly any one of compounds 1 to 193, as disclosed in U.S. Pat. No. 5,773,647 which is incorporated herein by reference, or a pharmaceutically acceptable salt or solvate thereof, such as a disodium salt, an ethanol solvate, or a hydrate of any one of these compounds), any of the nanoparticles described in WO 2011/133198, any of the polymer preparations described in US 2015/174076 and/or a hydrogel (e.g., as described in Torres-Lugo M et al. Biotechnol Prog. 2002; 18(3):612-6) can likewise be used as permeation enhancer. Moreover, a complex lipoidal dispersion (e.g., a combination of an insoluble surfactant or oil with a soluble surfactant, and optionally with water or a co-solvent) can also be used as permeation enhancer; corresponding exemplary permeation enhancers include, in particular, mixed micelles, reversed micelles, a self emulsifying system (e.g., SEDDS, SMEDDS, or SNEDDS), a lipid dispersion, a course emulsion, or solid lipid nanoparticles (SLNs). Preferably, the permeation enhancer is selected from sodium caprylate, sodium caprate, sodium laurate, sucrose laurate, sucrose stearate, sodium stearate, EDTA, polyacrylic acid, and N-[8-(2-hydroxybenzoyl)amino]caprylate or a pharmaceutically acceptable salt thereof (particularly sodium N-[8-(2-hydroxybenzoyl)amino]caprylate). A particularly preferred permeation enhancer is N-[8-(2-hydroxybenzoyl)amino]caprylate or a pharmaceutically acceptable salt thereof, in particular sodium N-[8-(2-hydroxybenzoyl)amino]caprylate. Moreover, if the pharmaceutical composition is for oral administration, it is particularly preferred that the permeation enhancer is sodium caprate.

Further preferred permeation enhancers are alky polysaccharides, arginine or CriticalSorb® (Solutol® HS15). In particular, the permeation enhancer may an alkyl glycoside (or a combination of two or more alkyl glycosides) which may be selected from any of the alkyl glycosides described in the following.

Alkyl glycosides to be used as permeation enhancer in accordance with the present invention can be synthesized by known procedures, i.e., chemically, as described, e.g., in Rosevear et al., Biochemistry 19:4108-4115 (1980) or Koeltzow and Urfer, J. Am. Oil Chem. Soc., 61:1651-1655 (1984), U.S. Pat. No. 3,219,656 or 3,839,318 or enzymatically, as described, e.g., in Li et al., J. Biol. Chem., 266:10723-10726 (1991) or Gopalan et al., J. Biol. Chem. 267:9629-9638 (1992).

Alkyl glycosides to be used as permeation enhancer in the present invention can include, but are not limited to: alkyl glycosides, such as octyl-, nonyl-, decyl-, undecyl-, dodecyl-, tridecyl-, tetradecyl-, pentadecyl-, hexadecyl-, heptadecyl-, and octadecyl-α- or β-D-maltoside, -glucoside or -sucroside (which may be synthesized according to Koeltzow and Urfer; Anatrace Inc., Maumee, Ohio; Calbiochem, San Diego, Calif.; Fluka Chemie, Switzerland); alkyl thiomaltosides, such as heptyl-, octyl-, dodecyl-, tridecyl-, and tetradecyl-β-D-thiomaltoside (which may be synthesized according to Defaye, J. and Pederson, C., “Hydrogen Fluoride, Solvent and Reagent for Carbohydrate Conversion Technology” in Carbohydrates as Organic Raw Materials, 247-265 (F. W. Lichtenthaler, ed.) VCH Publishers, New York (1991); Ferenci, T., J. Bacteriol, 144:7-11 (1980)); alkyl thioglucosides, such as heptyl- or octyl 1-thio α- or β-D-glucopyranoside (Anatrace, Inc., Maumee, Ohio; see Saito, S. and Tsuchiya, T. Chem. Pharm. Bull. 33:503-508 (1985)); alkyl thiosucroses (which may be synthesized according to, for example, Binder, T. P. and Robyt, J. F., Carbohydr. Res. 140:9-20 (1985)); alkyl maltotriosides (which may be synthesized according to Koeltzow and Urfer); long chain aliphatic carbonic acid amides of sucrose β-amino-alkyl ethers (which may be synthesized according to Austrian Patent 382,381 (1987); Chem. Abstr., 108:114719 (1988) and Gruber and Greber pp. 95-116); derivatives of palatinose and isomaltamine linked by amide linkage to an alkyl chain (which may be synthesized according to Kunz, M., “Sucrose-based Hydrophilic Building Blocks as Intermediates for the Synthesis of Surfactants and Polymers” in Carbohydrates as Organic Raw Materials, 127-153); derivatives of isomaltamine linked by urea to an alkyl chain (which may be synthesized according to Kunz); long chain aliphatic carbonic acid ureides of sucrose β-amino-alkyl ethers (which may be synthesized according to Gruber and Greber, pp. 95-116); and long chain aliphatic carbonic acid amides of sucrose β-amino-alkyl ethers (which may be synthesized according to Austrian Patent 382,381 (1987), Chem. Abstr., 108:114719 (1988) and Gruber and Greber, pp. 95-116).

The permeation enhancer may also be selected from any of the enhancing agents referred to in U.S. Pat. No. 8,927,497, including in particular any of alkyl glycosides, any of the saccharide alkyl esters, and/or any of the mucosal delivery-enhancing agents described in this document.

Moreover, the permeation enhancer may also be a compound of the following formula (I):

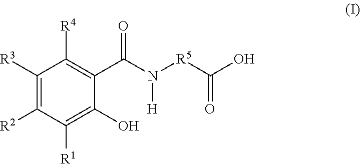

wherein:
R¹, R², R³ and R⁴ are each independently selected from hydrogen, —OH, —NR⁶R⁷, halogen (e.g., —F, —Cl, —Br or —I), C₁₋₄ alkyl or C₁₋₄ alkoxy;
R⁵ is a substituted or unsubstituted C₂₋₁₆ alkylene, substituted or unsubstituted C₂₋₁₆ alkenylene, substituted or unsubstituted C₁₋₁₂ alkyl(arylene) [e.g., substituted or unsubstituted C₁₋₁₂ alkyl(phenylene)], or substituted or unsubstituted aryl(C₁₋₁₂ alkylene) [e.g., substituted or unsubstituted phenyl(C₁₋₁₂ alkylene)]; and
R⁶ and R⁷ are each independently hydrogen, oxygen, —OH or C₁₋₄ alkyl;
or a pharmaceutically acceptable salt or solvate thereof, particularly a disodium salt, an alcohol solvate (e.g., a methanol solvate, an ethanol solvate, a propanol solvate, or a propylene glycol solvate, or any such solvate of the disodium salt; particularly an ethanol solvate or an ethanol solvate of the disodium salt), or a hydrate thereof (e.g., a monohydrate of the disodium salt). The above-mentioned “substituted” groups comprised in formula (I) are preferably substituted with one or more (e.g., one, two, or three) substituent groups independently selected from halogen (e.g., —F, —Cl, —Br or —I), —OH, C₁₋₄ alkyl or C₁₋₄ alkoxy. Such compounds and methods for their preparation are described, e.g., in WO 00/59863 which is incorporated herein by reference. Accordingly, the permeation enhancer may also be a “delivery agent” as described in WO 00/59863. Preferred examples of the compounds of formula (I) include N-(5-chlorosalicyloyl)-8-aminocaprylic acid, N-(10-[2-hydroxybenzoyl]amino)decanoic acid, N-(8-[2-hydroxybenzoyl]amino)caprylic acid, a monosodium or disodium salt of any one of the aforementioned compounds, an ethanol solvate of the sodium salt (e.g., monosodium or disodium salt) of any one of the aforementioned compounds, a monohydrate of the sodium salt (e.g., monosodium or disodium salt) of any one of the aforementioned compounds, and any combination thereof. A particularly preferred compound of formula (I) is the disodium salt of N-(5-chlorosalicyloyl)-8-aminocaprylic acid or the monohydrate thereof.

Furthermore, if the peptide or protein drug is GLP-1, a GLP-1 analog, a GLP-1 agonist, or a dual agonist of the GLP-1 receptor and another receptor (e.g., a dual agonist of the GLP-1 receptor and the glucagon receptor, or a dual agonist of the GLP-1 receptor and the gastric inhibitory polypeptide (GIP) receptor), then it is particularly preferred to use a permeation enhancer selected from sucrose laurate, sodium caprate, sodium chenodeoxycholate, a nd SNAC. If the peptide or protein drug is desmopressin, a desmopressin analog, or a vasopressin receptor 2 agonist peptide, then it is particularly preferred to use a permeation enhancer selected from SNAC, arginine, sucrose laurate, and sucrose stearate.

The pharmaceutical composition of the present invention may be, for example, a solid composition or a liquid composition. The solid composition is preferably a solid composition (e.g., a tablet or a powder) which is substantially water-free, e.g., contains less than about 5% (w/w) of water, preferably less than about 3% (w/w) of water, more preferably less than about 1% (w/w) of water, even more preferably less than about 0.5% (w/w) of water, yet even more preferably less than about 0.1% (w/w) of water, and is still more preferably free of water. The liquid composition may be, for instance, a liquid substantially water-free composition, such as, e.g., a liquid composition that contains less than about 5% (v/v) of water, or less than about 3% (v/v) of water, or less than about 1% (v/v) of water, or less than about 0.5% (v/v) of water, or less than about 0.1% (v/v) of water, or is free of water. Alternatively, the liquid composition may, e.g., be based on water, an oil, an organic solvent, or a mixture thereof; accordingly, the liquid composition may comprise, for example, at least about 60% (v/v) (or, e.g., at least about 70, 80 or 90% (v/v)) of water, an oil or an organic solvent, with respect to the total volume of the corresponding liquid composition. The organic solvent is not particularly limited, and is preferably selected from glycerol, propylene glycol (particularly propane-1,2-diol), and ethanol. The liquid composition may be, e.g., a solution, a suspension or an emulsion (such as an oil-in-water emulsion or a water-in-oil emulsion); in particular, the pharmaceutical composition is an aqueous composition (i.e., an aqueous liquid composition), such as an aqueous solution. The aqueous composition (or the aqueous solution) comprises water, preferably at least about 60% (v/v) water, more preferably at least about 70% (v/v) water, even more preferably at least about 80% (v/v) water, even more preferably at least about 90% (v/v) water, and yet even more preferably at least about 95% (v/v) water, with respect to the total volume of the corresponding (liquid) pharmaceutical composition. As explained above, the aqueous composition may be, e.g., an aqueous solution, an aqueous suspension or an oil-in-water emulsion; in this regard, it is preferred that the aqueous composition has an oil content of less than about 5% (v/v), more preferably of less than about 3% (v/v), even more preferably of less than about 2% (v/v), even more preferably of less than about 1% (v/v), even more preferably of less than about 0.5% (v/v), and yet even more preferably it does not contain any oil. Accordingly, it is preferred that the aqueous composition is an aqueous solution. It is furthermore preferred that the aqueous composition (or the aqueous solution) is isotonic with respect to human blood plasma. In particular, it is preferred that the aqueous composition (or the aqueous solution) has an osmolality of about 280 mOsm/kg to about 500 mOsm/kg, more preferably an osmolality of about 285 mOsm/kg to about 350 mOsm/kg, even more preferably an osmolality of about 290 mOsm/kg to about 300 mOsm/kg, and still more preferably an osmolality of about 296 mOsm/kg.

The pharmaceutical composition according to the present invention may also be a composition of a GLP-1 peptide, which composition is prepared as described in WO 2013/139694 but further comprises an excipient with a plc value of 12 or higher (as described and defined herein). Preferably, a salt of N-(8-(2-hydroxybenzoyl)amino)caprylic acid and the excipient with a pKₐ value of 12 or higher are present in the first type of granules, and the GLP-1 peptide is present in the second type of granules. Alternatively, a salt of N-(8-(2-hydroxybenzoyl)amino)caprylic acid is present in the first type of granules, and the excipient with a pKₐ value of 12 or higher as well as the GLP-1 peptide are present in the second type of granules.

Moreover, the pharmaceutical composition may also be in the form of a mucoadhesive product or device, such as a mucoadhesive patch or a liquid spray containing one or more mucoadhesive polymers, e.g., as described in US 2015/0174076, US 2003/0017195, or Ugwoke M I et al., Adv Drug Deliv Rev. 2005; 57(11):1640-65.

Furthermore, it is preferred that the peptide or protein drug is physically separated from the excipient with a pKₐ value of 12 or higher within the pharmaceutical composition according to the present invention. Accordingly, it is preferred that the pharmaceutical composition according to the invention is a pharmaceutical dosage form in which the peptide or protein drug is physically separated from the excipient with a pKₐ value of 12 or higher. A corresponding pharmaceutical dosage form preferably comprises at least two separate compartments which are physically separated from one another (e.g., through a physical separation layer). Accordingly, it is preferred that the pharmaceutical dosage form comprises a physical separation layer between (i) the peptide or protein drug and (ii) the excipient with a pKₐ value of 12 or higher. The peptide or protein drug is present only in a first compartment, and the excipient with a pKₐ value of 12 or higher is present only in a second compartment of the pharmaceutical dosage form. The permeation enhancer (if present) may be present either in the first compartment, or in the second compartment, or in both the first and the second compartment, or in a third compartment of the pharmaceutical dosage form. In one preferred embodiment, the invention thus provides a pharmaceutical dosage form (e.g., a double capsule) comprising: a peptide or protein drug, which is present in a first compartment of the pharmaceutical dosage form; an excipient with a pKₐ value of 12 or higher, which is present in a second compartment of the pharmaceutical dosage form; and optionally a permeation enhancer, which (if present) is in the first compartment and/or the second compartment of the pharmaceutical dosage form. In a further preferred embodiment, the invention provides a pharmaceutical dosage form (e.g., a multi-particulate dosage form) comprising: a peptide or protein drug, which is present in a first compartment of the pharmaceutical dosage form; an excipient with a pKₐ value of 12 or higher, which is present in a second compartment of the pharmaceutical dosage form; and optionally a permeation enhancer, which (if present) is in a third compartment of the pharmaceutical dosage form. It is particularly preferred that the pharmaceutical dosage form is a capsule inside a capsule (also referred to as a double capsule) or a multi-particulate dosage form. In the case of a double capsule, it is preferred that the bigger outer capsule (the content of which will be released first) contains the excipient with a pKₐ value of 12 or higher and optionally a permeation enhancer, and that the smaller inner capsule (the content of which will be released later) contains the peptide or protein drug. The dosage form may also be a release-modified dosage form, such as a dosage form (e.g., a capsule, multiparticulate or tablet) having an enteric coating or a dosage form (e.g., a capsule, multiparticulate or tablet) coated with Eudragit L30D55 or with Eudragit FS30D or an acid resistant capsule such as HPMCP capsules (commercially known as AR Caps®).

It is furthermore preferred that the pharmaceutical composition comprises particles of the excipient with a pKₐ value of 12 or higher, wherein said particles are coated with a protective coating that separates the excipient with a pKₐ value of 12 or higher from the peptide or protein drug. In particular, it is preferred that the pharmaceutical composition comprises particles of arginine free base, wherein said particles are coated with a protective coating that separates the arginine free base from the peptide or protein drug. The protective coating may, e.g., have a solubility in water of at least one gram per 100 ml of water at 20° C. Preferably, the protective coating is made of glucose, maltodextrin, or HPMC.

The peptide or protein drug may be first granulated with an inert pharmaceutical excipient and thereafter physically mixed with the excipient having a pKₐ value of 12 or higher (such as, e.g., arginine free base or trisodium phosphate). Preferably, the peptide or protein drug is first granulated with an inert pharmaceutical excipient and thereafter the granulate is coated with an additional pharmaceutical excipient to provide a physical separation between the peptide or protein drug and the excipient with a pKₐ value of 12 or higher.

The pharmaceutical composition (or the above-described pharmaceutical dosage form) may comprise the excipient with a pKₐ value of 12 or higher (including, in particular, any one or more of the above-mentioned exemplary excipients) in an amount of, e.g., about 1 mg to about 1000 mg per dosage unit, preferably in an amount of about 50 mg to about 500 mg per dosage unit. Moreover, if the pharmaceutical composition comprises a permeation enhancer, the permeation enhancer is preferably included in an amount of about 10 mg to about 1000 mg per dosage unit, more preferably about 50 mg to about 500 mg per dosage unit.

It is furthermore preferred that the constitution of the pharmaceutical composition (or the pharmaceutical dosage form) is such that, if the pharmaceutical composition is added to 10 ml of 0.1 M aqueous sodium bicarbonate (NaHCO₃) solution, the pH of the solution will be higher than pH 9, more preferably higher than pH 10, even more preferably higher than pH 11, or still more preferably higher than pH 12. Such a constitution of the pharmaceutical composition, resulting in the aforementioned pH, is advantageous as it allows a highly effective inactivation of proteolytic enzymes, as also demonstrated in Examples 1 and 2. In particular, the amount of the excipient with a pKₐ value of 12 or higher (and optionally the amount(s) of the peptide or protein drug and/or any further components comprised in the pharmaceutical composition) can be chosen such that, if the pharmaceutical composition is added to 10 ml of 0.1 M aqueous sodium bicarbonate solution, the pH of the solution will be higher than pH 9, more preferably higher than pH 10, even more preferably higher than pH 11, or still more preferably higher than pH 12.

The pharmaceutically acceptable salts referred to herein may be formed, e.g., by protonation of an atom carrying an electron lone pair which is susceptible to protonation, such as an amino group, with an inorganic or organic acid, or as a salt of a carboxylic acid group with a physiologically acceptable cation as they are well-known in the art. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts, nitrate salts, phosphate salts (such as, e.g., phosphate, hydrogenphosphate, or dihydrogenphosphate salts), carbonate salts, hydrogencarbonate salts or perchlorate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, glycolate, nicotinate, benzoate, salicylate, ascorbate, or pamoate (embonate) salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; and acidic amino acid salts such as aspartate or glutamate salts. It is to be understood that the term “pharmaceutically acceptable salt” also embraces pharmaceutically acceptable salts of the corresponding compound in any solvated form.

The peptide or protein drug, the excipient with a pKₐ value of 12 or higher, and the optionally used permeation enhancer can be formulated as a medicament, e.g., in the form of a pharmaceutical composition. The medicament or pharmaceutical composition may optionally comprise one or more further pharmaceutically acceptable excipients, such as carriers, diluents, fillers, disintegrants, lubricating agents, binders, colorants, pigments, stabilizers, preservatives, antioxidants, aminoacids, reducing agents, bioadhesive agents and/or solubility enhancers. In particular, it may comprise one or more additives selected from vitamin E, histidine, microcrystalline cellulose (MCC), mannitol, starch, sorbitol and/or lactose. The pharmaceutical composition can be formulated by techniques known to the person skilled in the art, such as the techniques published in Remington's Pharmaceutical Sciences, 20^{th} Edition.

As noted above, the pharmaceutical composition may comprise one or more solubility enhancers, such as, e.g., poly(ethylene glycol), including poly(ethylene glycol) having a molecular weight in the range of about 200 to about 5,000 Da, ethylene glycol, propylene glycol, non-ionic surfactants, tyloxapol, polysorbate 20, polysorbate 80, macrogol-15-hydroxystearate, phospholipids, lecithin, dimyristoyl phosphatidylcholine, dipalmitoyl phosphatidylcholine, distearoyl phosphatidylcholine, cyclodextrins, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioethers, hydroxypropyl methylcellulose, hydroxypropylcellulose, polyvinylpyrrolidone, vinyl acetate copolymers, vinyl pyrrolidone, sodium lauryl sulfate, dioctyl sodium suifosuccinate, or any combination thereof. Preferably, the one or more solubility enhancers include at least one non-ionic surfactant, more preferably at least one non-ionic surfactant having a hydrophilic-lipophilic balance (HLB) of greater than 10 (i.e., HLB>10). The pharmaceutical composition may also comprise at least one non-ionic surfactant having an HLB>10 and at least one non-ionic surfactant having an HLB<10.

It is thus preferred that the pharmaceutical composition comprises at least one non-ionic surfactant. In particular, the pharmaceutical composition may comprise a substance (preferably a detergent) that is capable of adsorbing at surfaces and/or interfaces (such as liquid to air, liquid to liquid, liquid to container, or liquid to any solid) and that has no charged groups in its hydrophilic group(s) (sometimes referred to as “heads”). The non-ionic surfactant may be a detergent and may, in particular, be selected from ethoxylated castor oil, a polyglycolyzed glyceride, an acetylated monoglyceride, a sorbitan-fatty-acid-ester, a polysorbate (such as, e.g., polysorbate-20, polysorbate-40, polysorbate-60, polysorbate-80, super-refined polysorbate 20, super-refined polysorbate 40, super-refined polysorbate 60, or super-refined polysorbate 80; including any of the corresponding Tween products, e.g., from the supplier Croda), a poloxamer (such as, e.g., poloxamer 188 or poloxamer 407), a polyoxyethylene sorbitan fatty acid ester, a polyoxyethylene derivative (such as, e.g., an alkylated and/or alkoxylated polyoxyethylene derivative; particularly a Tween product like, e.g., Tween-20 or Tween-80), a block copolymer such as, e.g., a polyethyleneoxide/polypropyleneoxide block copolymer (e.g., Pluronics/Tetronics, TritonX-100 and/or Synperonic PE/L44PEL), an ethoxylated sorbitan alkanoate (such as, e.g., Tween-20, Tween-40, Tween-80, or Brij-35), diglycerol laurate, diglycerol caprate, diglycerol caprylate, diglycerol monocaprylate, polyglycerol laurate, polyglycerol caprate, polyglycerol caprylate, or any combination thereof. Further examples of non-ionic surfactants that may be used as solubility enhancers in accordance with the invention include, but are not limited to: (1.) reaction products of a natural or hydrogenated castor oil and ethylene oxide (where the natural or hydrogenated castor oil may be reacted with ethyleneoxide in a molar ratio of from about 1:35 to about 1:60, with optional removal of the PEG component from the products; various such surfactants are commercially available, e.g., the CREMOPHOR series from BASF Corp. (Mt. Olive, N.J.), such as CREMOPHOR RH 40 which is PEG40 hydrogenated castor oil and an HLB of about 14-16); (2.) polyoxyethylene fatty acid esters, including in particular polyoxyethylene stearic acid esters (such as the MYRJ series from Uniqema, e.g., MYRJ 53 having a m.p. of about 47° C.; particular compounds in the MYRJ series are, e.g., MYRJ 53 having a m.p. of about 47° C. and PEG-40-stearate which is available, e.g., as MYRJ 52); (3.) sorbitan derivatives, including in particular the TWEEN series from Uniqema (e.g., TWEEN 60, Tween 20, Tween 80, or Tween 40); (4.) polyoxyethylene-polyoxypropylene co-polymers and/or block co-polymers and/or poloxamers (e.g., Pluronic P127 or Pluronic F68 from BASF or Synperonic PE/L from Croda); (5.) polyoxyethylenealkylethers (such as, e.g., polyoxyethylene glycol ethers of C12-C18 alcohols, like, e.g., polyoxyl 10- or 20-cetylether or polyoxyl 23-laurylether, or 20-oleylether, or polyoxyl 10-, 20- or 100-stearylether, e.g., as commercially available as the BRI series from Uniqema; particularly useful products from the BRIJ series include BRIJ 58, BRIJ 76, BRIJ 78, BRIJ 35 (or polyoxyl 23-laurylether), or BRIJ 98 (or polyoxyl 20 oleyl ether); these products may have a m.p. between about 32° C. and about 43° C.); (6.) water-soluble tocopheryl PEG succinic acid esters (e.g., as available from Eastman Chemical Co., with a m.p. of about 36° C., such as, e.g, TPGS, particularly vitamin E-TPGS); (7.) PEG sterol ethers (such as, e.g., SOLULAN C24 (Choleth-24 and Cetheth-24) from Chemron (Paso Robles, Calif.); similar products which may also be used are those which are known and commercially available as NIKKOL BPS-30 (poly ethoxylated 30 phytosterol) and NIKKOL BPSH-25 (poly ethoxylated 25 phytostanol) from Nikko Chemicals); (8.) polyglycerol fatty acid esters, e.g., having 4 to 10 glycerol units, such as 4, 6 or 10 glycerol units (e.g., particularly suitable are deca-/hexa-/tetraglycerylmonostearate, e.g., DECAGLYN, HEXAGLYN or TETRAGLYN from Nikko Chemicals); (9.) alkylene polyolether or ester (e.g., lauroyl macrogol-32 glycerides and/or stearoylmacrogol-32 glycerides, such as GELUCIRE 44/14 and/or GELUCIRE 50/13); (10.) polyoxyethylenemonoesters of a saturated C₁₀-C₂₂ (e.g., C₁₈) hydroxy fatty acid (which may optionally be substituted), such as, e.g., 12-hydroxystearic acid PEG ester, e.g., of PEG 600, 900 or 660 (e.g., SOLUTOL HS 15 from BASF (Ludwigshafen, Germany); or a substance comprising (or consisting of) about 70% polyethoxylated 12-hydroxystearate by weight and about 30% by weight unesterified polyethylene glycol component, having a hydrogenation value of 90 to 110, a saponine cation value of 53 to 63, an acid number of maximum 1, and a maximum water content of 0.5% by weight); (11.) polyoxyethylene-polyoxypropylene-alkyl ethers (such as, e.g., polyoxyethylene-polyoxypropylene ethers of C₁₂-C₁₈ alcohols, e.g., polyoxyethylen-20-polyoxypropylene-4-cetylether, which is commercially available as NIKKOL PBC 34 from Nikko Chemicals); or (12.) polyethoxylated distearates (e.g., as commercially available under the trade names ATLAS G 1821 from Uniqema and/or NIKKOCDS-6000P from Nikko Chemicals).

Moreover, as noted above, the pharmaceutical composition may comprise one or more pharmaceutically acceptable carriers. The pharmaceutically acceptable carrier may be an aqueous or non-aqueous agent, for example alcoholic or oleaginous, or a mixture thereof, and may contain a surfactant, an emollient, a lubricant, a stabilizer, a dye, a perfume, a preservative, an acid or base for adjustment of pH, a solvent, an emulsifier, a gelling agent, a moisturizer, a stabilizer, a wetting agent, a time release agent, a humectant, or any other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, and oils such as olive oil or injectable organic esters. A pharmaceutically acceptable carrier can contain physiologically acceptable compounds that act, for example, to stabilize or to increase the absorption of the corresponding peptide or protein drug, for example, carbohydrates, such as glucose, sucrose or dextrans, antioxidants, such as ascorbic acid or glutathione, chelating agents, low molecular weight proteins or other stabilizers or excipients. A pharmaceutically acceptable carrier can also be selected from substances such as distilled water, benzyl alcohol, lactose, starches, talc, magnesium stearate, polyvinylpyrrolidone, alginic acid, colloidal silica, titanium dioxide, and flavoring agents.

It is furthermore preferred that the pharmaceutical composition according to the present invention is free of salts or complexes of copper, zinc or iron. Accordingly, it is preferred that the pharmaceutical composition does not contain any copper salts or complexes, any zinc salts or complexes, or any iron salts or complexes.

The pharmaceutical composition is formulated as a dosage form for transmucosal administration, preferably for oral administration, oromucosal administration, or nasal administration. Accordingly, it is preferred that the pharmaceutical composition is administered to a subject/patient transmucosally, particularly orally, oromucosally, or nasally. Preferably, the pharmaceutical composition is formulated as an oral dosage form, and is thus preferably administered orally.

For oral administration, the pharmaceutical composition is to be administered by oral ingestion, particularly by swallowing. The pharmaceutical composition can thus be administered to pass through the mouth into the gastrointestinal tract, which is also referred to as “oral-gastrointestinal” administration; in that case, the peptide or protein drug contained in the pharmaceutical composition can be absorbed through the gastric and/or intestinal mucosa. Oral administration also specifically includes oral-intestinal administration and/or oral-gastric administration.

Dosage forms for oral administration include, e.g., tablets (e.g., coated or uncoated tablets), capsules (e.g., HPMC capsules or HPMCP capsules), a capsule inside a capsule, mini patch systems inside a capsule, lozenges, troches, ovules, solutions, emulsions, suspensions, syrups, elixirs, powders and granules for reconstitution, dispersible powders and granules, medicated gums, chewing tablets, effervescent tablets, and multiparticulate dosage forms.

The tablets may contain excipients such as non-reducing sugars, microcrystalline cellulose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose and acacia. Additionally, lubricating agents such as magnesium stearate, stearic acid, glyceryl behenate and talc may be included. Solid compositions of a similar type may also be employed as fillers in hard capsules. Preferred excipients in this regard include non-reducing sugars, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

The pharmaceutical composition can thus be provided in the form of a tablet, e.g., a slow disintegrating tablet or slow eroding tablet. In particular, the pharmaceutical composition may be provided as a dosage form (e.g., a tablet) having an enteric coating, preferably an enteric coating that dissolves at a pH above 7.

For nasal administration, the pharmaceutical composition may be provided, e.g., as a nasal spray, as nasal drops, as an aerosol, or as a dry powder for nasal administration, particularly as a nasal spray. Nasal administration includes, in particular, intranasal transmucosal administration, local administration to the nasal cavity, or nose-to-brain delivery. Thus, the nasal administration of the pharmaceutical composition of the invention can have systemic therapeutic effects (particularly via absorption of the peptide or protein drug through the nasal mucosa) and/or local therapeutic effects (particularly in the nasal cavity) and/or therapeutic effects in the brain (particularly via nose-to-brain delivery; see, e.g., Kamble M S et al., International Journal of Pharmaceutical and Chemical Sciences. 2013; 2(1):516-25), depending inter alia on the choice of the specific peptide or protein drug to be administered and the optional use of a permeation enhancer or a mucoadhesive polymer.

The pharmaceutical composition of the present invention may also be administered oromucosally. Accordingly, the pharmaceutical composition may be formulated as a dosage form for oromucosal administration. Oromucosal administration refers to the deposition or application of the pharmaceutical composition onto a mucosal epithelium in the oral cavity of a subject/patient, such as the buccal, gingival, sublingual, palatal, sublabial, or oropharyngeal mucosal epithelium. Oromucosal administration thus includes, in particular, buccal administration, gingival administration, sublingual administration, palatal administration, sublabial administration, or oropharyngeal administration. Thus, the pharmaceutical composition may be administered, e.g., buccally, sublingually, gingivally, sublabially, or oropharyngeally.

For oromucosal administration, the pharmaceutical composition of the invention may be administered using any suitable oromucosal dosage form, such as, e.g., in the form of drops, as a spray, or by means of a dosage device (e.g., a spray device, a drop device, a unit dose device/dispenser, a multi-dose device/dispenser or ampoule, a dosage pen, or a dosage pipette). The dosage device may be fitted with an actuator and/or a discharge orifice to enable the patient or a carer to deposit the desired dose accurately within the oral cavity. The dosage device may be adapted to dispense, upon actuation, a predetermined volume (corresponding to a unit dose) of the pharmaceutical composition, e.g., in the form of a spray or in the form of one or more drops. The dosage device may also be a dosage pen which delivers a fixed volume containing a fixed dose of the peptide or protein drug.

Typically, a physician will determine the actual dosage which will be most suitable for an individual subject. The specific dose level and frequency of dosage for any particular individual subject may be varied and will depend upon a variety of factors including the activity of the specific peptide or protein drug employed, the metabolic stability and length of action of that compound, the age, body weight, general health, sex, diet, mode and time of administration, rate of excretion, drug combination, the severity of the particular condition, and the individual subject undergoing therapy. The precise dose will ultimately be at the discretion of the attendant physician or veterinarian.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a disorder or disease as used herein is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alga, comprise curative treatment (preferably leading to a complete response and eventually to healing of the disorder or disease) and palliative treatment (including symptomatic relief).

The term “prevention” of a disorder or disease as used herein is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a peptide or protein drug according to the invention before any clinical and/or pathological symptoms are diagnosed or determined or can be diagnosed or determined by the attending physician.

The terms “peptide” and “protein”, as in the expression “peptide or protein drug”, are used herein interchangeably and refer to a polymer of two or more amino acids linked via amide bonds that are formed between an amino group of one amino acid and a carboxyl group of another amino acid. The amino acids comprised in the peptide or protein, which are also referred to as amino acid residues, may be selected from the 20 standard proteinogenic α-amino acids (i.e., Ala, Arg, Asn, Asp, Cys, Glu, Gln, Gly, His, Ile, Leu, Lys, Met, Phe, Pro, Ser, Thr, Trp, Tyr, and Val) but also from non-proteinogenic and/or non-standard α-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine) as well as β-amino acids (e.g., β-alanine), γ-amino acids (e.g., γ-aminobutyric acid, isoglutamine, or statine) and δ-amino acids. Preferably, the amino acid residues comprised in the peptide or protein are selected from α-amino acids, more preferably from the 20 standard proteinogenic α-amino acids (which can be present as the L-isomer or the D-isomer, and are preferably all present as the L-isomer). The peptide or protein may be unmodified or may be modified, e.g., at its N-terminus, at its C-terminus and/or at a functional group in the side chain of any of its amino acid residues (particularly at the side chain functional group of one or more Lys, His, Ser, Thr, Tyr, Cys, Asp, Glu, and/or Arg residues). Such modifications may include, e.g., the attachment of any of the protecting groups described for the corresponding functional groups in: Wuts P G & Greene T W, Greene's protective groups in organic synthesis, John Wiley & Sons, 2006. Such modifications may also include the covalent attachment of one or more polyethylene glycol (PEG) chains (forming a PEGylated peptide or protein), the glycosylation and/or the acylation with one or more fatty acids (e.g., one or more C₈₋₃₀ alkanoic or alkenoic acids; forming a fatty acid acylated peptide or protein). Moreover, such modified peptides or proteins may also include peptidomimetics, provided that they contain at least two amino acids that are linked via an amide bond (formed between an amino group of one amino acid and a carboxyl group of another amino acid). The amino acid residues comprised in the peptide or protein may, e.g., be present as a linear molecular chain (forming a linear peptide or protein) or may form one or more rings (corresponding to a cyclic peptide or protein). The peptide or protein may also form oligomers consisting of two or more identical or different molecules.

The term “amino acid” refers, in particular, to any one of the 20 standard proteinogenic α-amino acids (i.e., Ala, Arg, Asn, Asp, Cys, Glu, Gln, Gly, His, Ile, Leu, Lys, Met, Phe, Pro, Ser, Thr, Trp, Tyr, and Val) but also to non-proteinogenic and/or non-standard α-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine) as well as β-amino acids (e.g., β-alanine), γ-amino acids (e.g., γ-aminobutyric acid, isoglutamine, or statine) and/or δ-amino acids as well as any other compound comprising at least one carboxylic acid group and at least one amino group. Unless defined otherwise, an “amino acid” preferably refers to an α-amino acid, more preferably to any one of the 20 standard proteinogenic α-amino acids (which can be present as the L-isomer or the D-isomer, and are preferably present as the L-isomer).

The term “antibody” refers to any immunoglobulin molecule that specifically binds to (or is immunologically reactive with) a particular antigen. The antibody may be, e.g., a monoclonal antibody or a polyclonal antibody, and is preferably a monoclonal antibody. Moreover, the antibody (e.g., the monoclonal antibody) may be a whole antibody (e.g., IgA, IgD, IgE, IgM or IgG, including in particular IgG1, IgG2, IgG3 or IgG4), a chimeric antibody, a humanized antibody, a human antibody, a heteroconjugate antibody (e.g., a bispecific antibody), or it may be an antigen-binding fragment of any of the aforementioned types of antibody (such as, e.g., Fab, Fab′, F(ab′)₂, Fv, or scFv). The antibody may also be a single-chain antibody (scAb) or a single-domain antibody (sdAb; e.g., a “nanobody”).

The term “complex” refers to a chelate complex (in which coordinate bonds are formed between a single central atom/ion and a polydentate ligand) or a coordination complex composed of monodentate ligands coordinating a single central atom/ion.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, if a component of a composition is indicated to be “optional”, the invention specifically relates to both possibilities, i.e., that the corresponding component is present (contained in the composition) or that the corresponding component is absent from the composition.

As used herein, the term “about” refers to ±10% of the indicated numerical value, preferably to ±5% of the indicated numerical value, and in particular to the exact numerical value indicated. For example, the expression “about 100” refers to the range of 90 to 110, in particular the range of 95 to 105, and preferably refers to the specific value of 100. If the term “about” is used in connection with the endpoints of a range, it refers to the range from the lower endpoint −10% of its indicated numerical value to the upper endpoint +10% of its indicated numerical value, in particular to the range from of the lower endpoint −5% to the upper endpoint +5%, and preferably to the range defined by the exact numerical values of the lower endpoint and the upper endpoint. Thus, the expression “about 10 to about 20” refers to the range of 9 to 22, in particular 9.5 to 21, and preferably 10 to 20. If the term “about” is used in connection with the endpoint of an open-ended range, it refers to the corresponding range starting from the lower endpoint −10% or from the upper endpoint +10%, in particular to the range starting from the lower endpoint −5% or from the upper endpoint +5%, and preferably to the open-ended range defined by the exact numerical value of the corresponding endpoint. For example, the expression “at least about 10%” refers to at least 9%, particularly at least 9.5%, and preferably at least 10%.

Unless specifically indicated otherwise, all properties and parameters referred to herein (including, e.g., any amounts/concentrations indicated in “mg/ml” or in “% (v/v)”, and any pH values) are preferably to be determined at standard ambient temperature and pressure conditions, particularly at a temperature of 25° C. (298.15 K) and at an absolute pressure of 101.325 kPa (1 atm).

Furthermore, it is to be understood that the present invention specifically relates to each and every combination of features and embodiments described herein, including any combination of general and/or preferred features/embodiments. In particular, the invention specifically relates to all combinations of preferred features described herein.

In this specification, a number of documents including patents, patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention is also described by the following illustrative figures. The appended figures show:

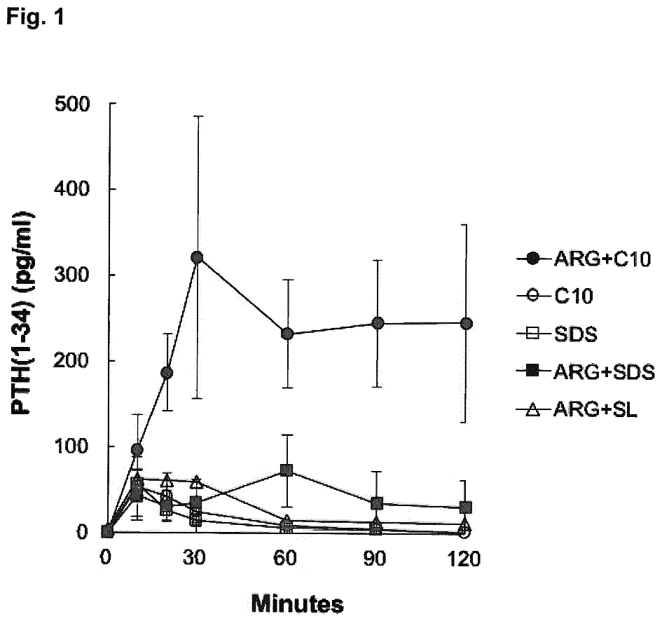

FIG. 1: Pharmacokinetic profile of PTH(1-34) formulations after intestinal administration in pigs (see Example 4). Formulations comprising: -●- L-arginine free base and sodium caprate (“ARG+C10”; n=3), -▪- L-arginine free base and sodium dodecyl sulfate (“ARG+SDS”; n=3), -Δ- L-arginine free base and sucrose laurate (“ARG+SL”; n=1), -□- sodium dodecyl sulfate (“SDS”; n=4), -∘- sodium caprate (“C10”; n=3).

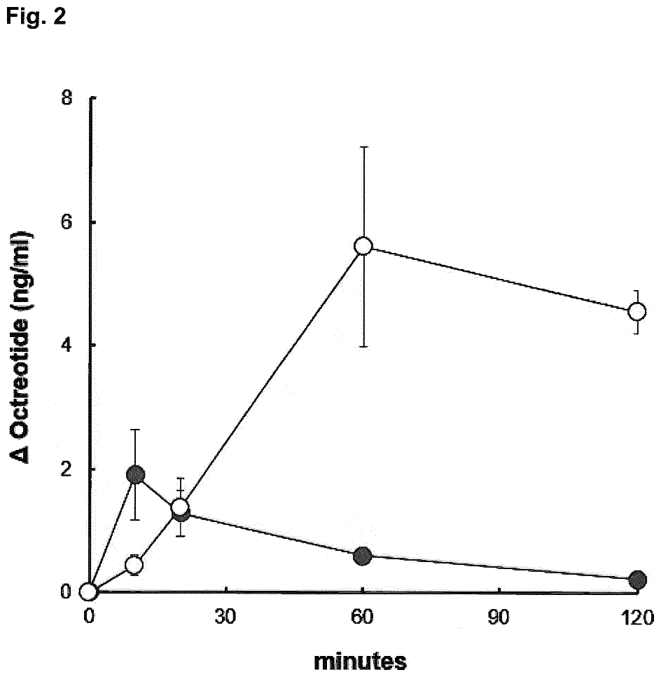

FIG. 2: Plasma octreotide concentration after intestinal injection in rats (see Example 11); OCT005 (-●-), OCT006 (-∘-); each data point represents the mean of at least n=3±S.E.

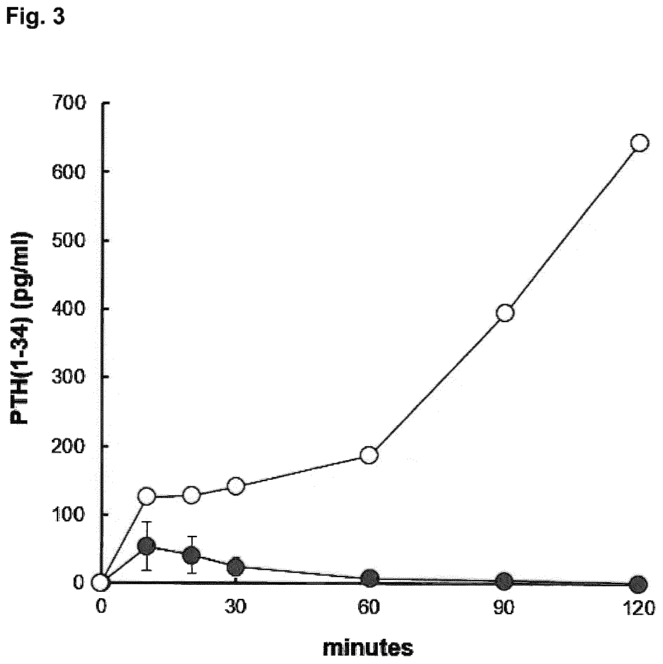

FIG. 3: Plasma PTH(1-34) concentration after intestinal application in pigs (see Example 12); sodium caprate in solution (-●-; n=3±S.E.), matrix tablet with L-arginine free base, sodium caprylate and polyacrylate (-∘-; n=1).

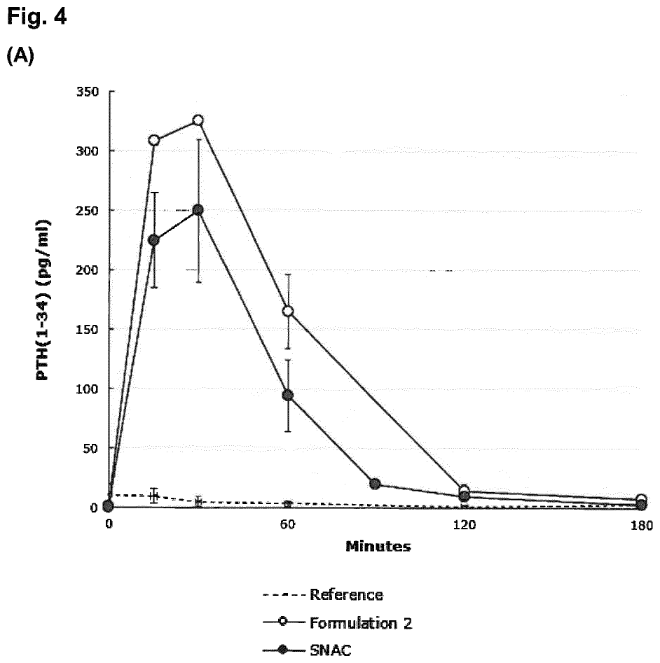

FIG. 4: (A) Pharmacokinetic profile of PTH(1-34) formulations after oral administration to non-human primates (see Example 15). (B) Individual pharmacokinetic profiles of PTH(1-34) formulations after oral administration to non-human primates.

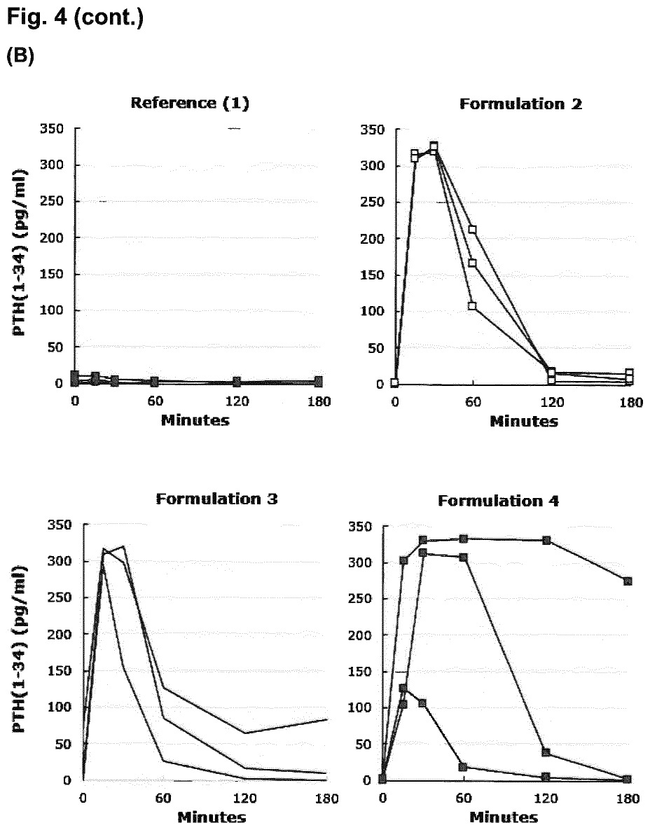

FIG. 5: Permeation of semaglutide across human nasal cell line RPMI 2650 (see Example 21).

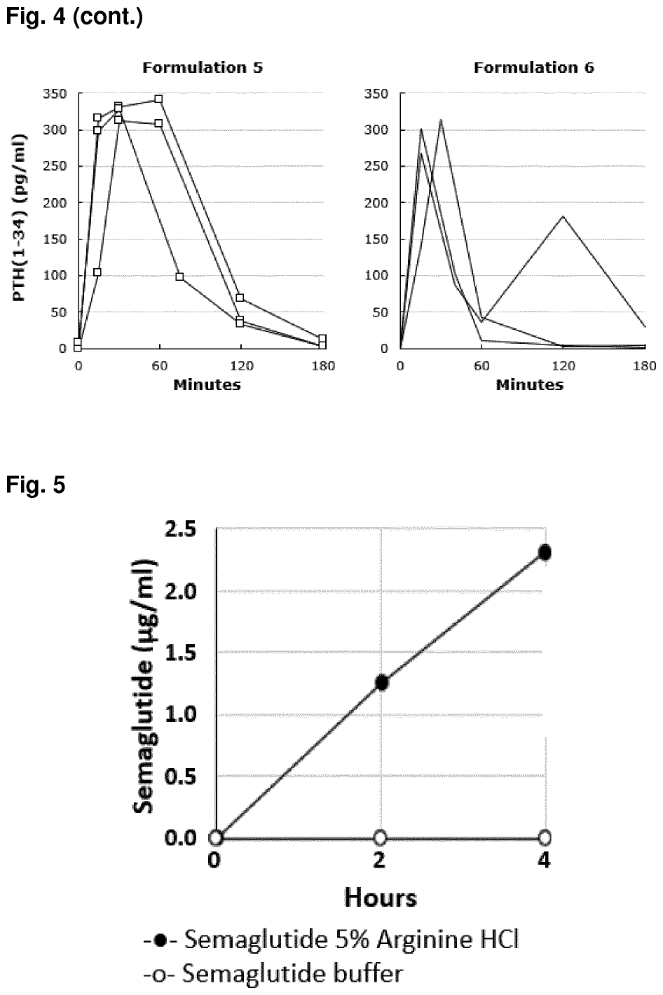

FIG. 6: Cell proliferation cytotoxicity Assay (MTS) (see Example 22).

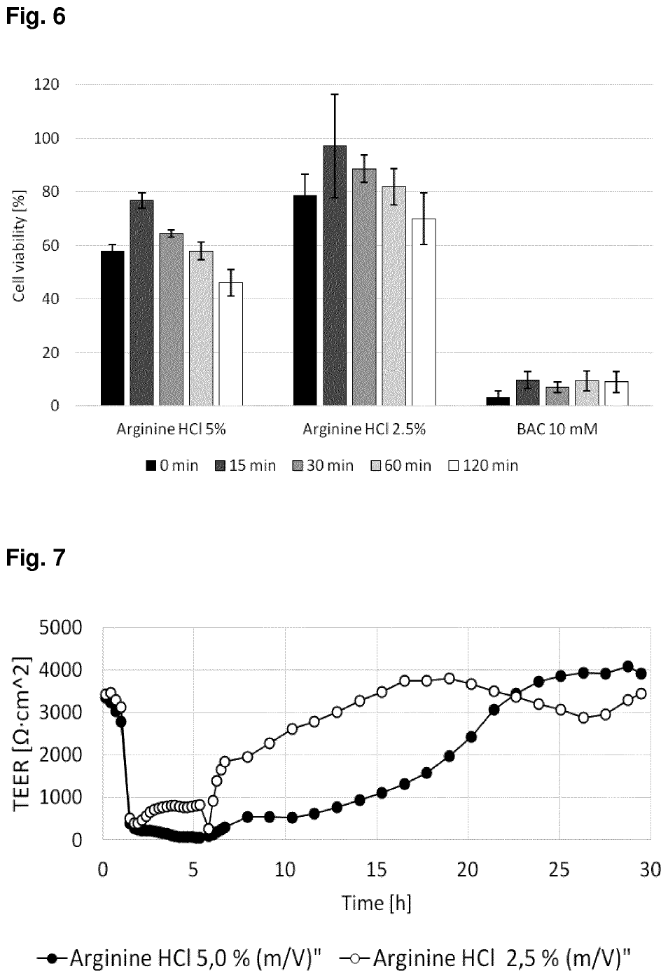

FIG. 7: TEER measurement in MDCK cells in presence of arginine hydrochloride (see Example 23).

The invention will now be described by reference to the following examples which are merely illustrative and are not to be construed as a limitation of the scope of the present invention.

### EXAMPLES

### Example 1: Stability of PTH(1-34) Formulations in Simulated Gastric Fluid Containing Pepsin (SGFP)

PTH(1-34) formulations were incubated for 20 minutes at 37° C. in simulated gastric fluid comprising a final concentration of 1.6 mg/ml pepsin. Intact PTH(1-34) was analyzed by HPLC. The results are shown in Table 1:

TABLE 1

| | | | | Intact |
|---|---|---|---|---|
| | PTH(1-34) | Inhibitor | pH | PTH(1-34) |
| PTH-SGFP-001 | 0.5 mg/ml | — | 1.6 | 0% |
| | | | (solution) | |
| PTH-SGFP-002 | 0.5 mg/ml | 7.5 mg/ml | 7.8 | 94% |
| | | L-Arginine | (solution) | |
| | | free base | | |
| PTH-SGFP-003 | 0.5 mg/ml | 7.5 mg/ml | 2.1 | 0% |
| | | L-Arginine | (solution) | |
| | | HCl | | |
| PTH-SGFP-004 | 0.5 mg/ml | 70 mg/ml | 6.2 | 29% |
| | | Zinc arginate | (slight | |
| | | chelate | suspension) | |
| PTH-SGFP-005 | 0.5 mg/ml | 7.5 mg/ml | 2.3 | 0% |
| | | SNAC | (suspension) | |
| PTH-SGFP-006 | 0.5 mg/ml | 7.5 mg/ml | 7.3 | 100% |
| | | SNAC + | (slight | |
| | | 7.5 mg/ml | suspension) | |
| | | L-Arginine | | |
| | | free base | | |

(SNAC: sodium N-[8-(2-hydroxybenzoyl)amino]caprylate)
Conclusion: Formulations comprising L-arginine free base were shown to prevent PTH(1-34) from degradation by pepsin in simulated gastric fluid. L-Arginine HCl, however, did not prevent degradation of the peptide in SGFP. The addition of zinc arginate chelate resulted in partial protection against pepsin degradation.

### Example 2: Stability of PTH(1-34) Formulations in Presence of Trypsin

PTH(1-34) formulations were incubated for 15 minutes at 37° C. in solutions comprising a final concentration of 0.05 mg/ml trypsin. Intact PTH(1-34) was analyzed by HPLC. The results are shown in Table 2.

TABLE 2

| | | Inhibitor | | Intact |
|---|---|---|---|---|
| | PTH(1-34) | (100 mg/ml) | pH | PTH(1-34) |
| PTH-TRY-001 | 0.5 mg/ml | — | 6.4 | 0% |
| PTH-TRY-002 | 0.5 mg/ml | L-Arginine free base | 10.8 | 81.8% |
| PTH-TRY-003 | 0.5 mg/ml | L-Arginine HCl | 5.7 | 40.8% |

Conclusion: Formulations comprising L-arginine free base were shown to prevent PTH(1-34) from degradation by trypsin. L-Arginine HCl prevented degradation of the peptide in presence of trypsin to a lesser extent.

### Example 3: Stability of PTH(1-34) Formulations in Presence of Porcine Nasal Mucosa

Formulations with PTH(1-34):

### PTH-NAS-001

0.5 mg/ml PTH(1-34) were dissolved in 0.5 M phosphate buffer (pH 7.4).

### PTH-NAS-002

0.5 mg/ml PTH(1-34) and 100 mg/ml L-arginine free base were dissolved in 0.5 M phosphate buffer (pH 7.4).

### PTH-NAS-003

0.5 mg/ml PTH(1-34) and 100 mg/ml L-arginine HCl were dissolved in 0.5 M phosphate buffer (pH 7.4).

PTH(1-34) formulations were incubated for 4 hours at 37° C. with excised porcine nasal mucosa (50 mg/ml). Intact PTH(1-34) was analyzed by HPLC. The results are shown in Table 3.

TABLE 3

| | | Inhibitor | Intact |
|---|---|---|---|
| | PTH(1-34) | (100 mg/ml) | PTH(1-34) |
| PTH-NAS-001 | 0.5 mg/ml | — | 0% |
| PTH-NAS-002 | 0.5 mg/ml | L-Arginine base | 49.8% |
| PTH-NAS-003 | 0.5 mg/ml | L-Arginine HCl | 0% |

Conclusion: A nasal formulation comprising L-arginine substantially improved the enzymatic stability of PTH(1-34) in presence of nasal mucosa.

### Example 4: Pharmacokinetic Profile of PTH(1-34) Formulations after Intestinal Administration to Pigs

Formulations with PTH(1-34):

### PTH-PIG-001

### 2 mg PTH(1-34)

### 200 mg SDS

### PTH-PIG-002

### 2 mg PTH(1-34)

### 200 mg SDS

### 200 mg L-Arginine

### PTH-PIG-003

### 2 mg PTH(1-34)

200 mg Sodium caprate

### PTH-PIG-004

### 2 mg PTH(1-34)

200 mg Sodium caprate

### 200 mg L-Arginine

### PTH-PIG-005

### 2 mg PTH(1-34)

### 200 mg Sucrose Laurate

### 200 mg L-Arginine

The formulations were prepared as dry powder and dissolved in 2 ml H₂O 5 min prior to administration. The PTH(1-34) formulations were dosed into the distal jejunum in volume of 2 ml/pig (final concentration 1 mg/ml) to anaesthetized pigs. Blood was taken at the time points 0 prior dosing and at 10, 20, 30, 60, 90 and 120 min after dosing. Blood was drawn into Vacuette EDTA Aprotinin tubes (Greiner Bio-One). Blood samples were centrifuged (4 min, 10,000 g, 4° C.) and the PTH(1-34) plasma concentrations were determined using a commercial High Sensitivity Human PTH(1-34) ELISA kit (Immundiagnostik AG, Germany, KI603900). The data are expressed as mean±SE. The results are shown in FIG. 1.

Conclusion: Oral formulations comprising L-arginine and a permeation enhancer resulted in improved intestinal absorption of PTH(1-34). Especially the combination of L-arginine free base with sodium caprate resulted in surprisingly improved intestinal absorption of PTH(1-34). Such a formulation improved the AUC by more than 20-fold in comparison to compostions without L-arginine free base.

### Example 5: Pharmacokinetic Profile of Liraglutide in the Rat after Intestinal Administration

Formulation with Liraglutide:

### LIRA-INT-001

6 mg/ml Liraglutide
60 mg/ml L-Arginine
10 mg/ml Fe(II)gluconate
4 mg/ml Cu(II)gluconate
Final pH=9.7

The formulation was prepared as dry powder and dissolved 5 min prior administration. The liraglutide formulation was dosed into ileum in volume of 0.4 ml/kg (final concentration 6 mg/ml) to anaesthetized rats. Blood was taken from tail vessels at the time points 0, 30, 60, 90, 120, 180 and 240 min after dosing. Blood was drawn from the tail tip into Microtainer Microgard EDTA tubes (Becton Dickinson, USA). Blood samples were centrifuged (4 min, 10,000 g, 4° C.) and the liraglutide plasma concentrations were determined using commercial liraglutide EIA kit (Peninsula Laboratories International, USA, cat. number S-1502.0001). The data are expressed as mean±SE. The mean pharmacokinetic parameters are summarized in Table 4.

TABLE 4

| | | Adminis- | AUC(0-t) | Cmax |
|---|---|---|---|---|
| | Dose | tration | (ng/ml × min) | (ng/ml) |
| LIRA-INT- | 2.4 mg/kg | Rat Ileum | 58 964 ± 35 073 | 370.9 ± 244.6 |
| 001 | | | | |

Conclusion: A formulation comprising L-arginine resulted in a pronounced PK-profile of liraglutide after administration into the rat ileum.

### Example 6: In Vitro Permeation of Desmopressin Through Caco-2 Cell Monolayers

Permeation experiments with a ready to use Caco-2 cell kit were performed (CacoReady™, Readycell). The kit consisted of 24 insert-integrated permeable supports seeded with differentiated and polarized Caco-2 barriers on polycarbonate microporous filters. Cells were treated according to the instructions provided by the supplier. A desmopressin stock solution containing 2.83 mg of desmopressin per ml in simulated intestinal fluid (SIF pH 7, USP) was prepared. Further stock solutions were Arginine HCl, 1% in SIF pH 7, Arginine base, 1% in SIF pH 7 and trisodium phosphate, 1% in SIF pH 7. To each basolateral compartment, 0.75 ml of SIF pH 7, USP, was added. Four groups (with n=3 each) were tested. To the apical side of the permeation device, the following combinations were added: (1) 0.125 ml of desmopressin stock solution+0.125 ml of SIF, pH 7, USP; (2) 0.125 ml of desmopressin stock solution+0.125 ml of Arginine HCl 1% stock solution; (3) 0.125 ml of desmopressin stock solution+0.125 ml of Arginine free base 1% stock solution; and (4) 0.125 ml of desmopressin stock solution+0.125 ml of trisodium phosphate 1% stock solution. Subsequent to the addition of desmopressin to the apical side, the cells were incubated for 2 hours at 37° C., 5% CO₂ and high relative humidity. After incubation, the apical and basolateral contents of each experiment were collected and stored at −20° C., prior to analysis. The desmopressin concentration of the basolateral compartments was analysed via a gradient HPLC method, based on H₂O containing 0.1% trifluoroacetic acid and acetonitrile containing 0.1% trifluoroacetic acid.

Results: It has been shown that at 0.5% (m/v), Arginine HCl did not increase the transport of desmopressin through the Caco-2 monolayers compared with desmopressin in buffer only. However, the presence of 0.5% (m/v) of Arginine free base or of trisodium phosphate led to a significant increase of desmopressin permeation compared to the control in buffer only. The corresponding results are detailed in Table 5.

TABLE 5
Basolateral concentration of desmopressin (μg/ml) after
permeation through Caco-2 cell monolayers in presence
of 0.5% of different excipients (2 hours of incubation
at 37° C.); means of n = 3 ± S.D.

| | Desmopressin | | |
|---|---|---|---|
| | (μg/ml) | S.D. | Improvement |
| Apical medium | basolateral | (μg/ml) | factor |
| SIF, USP | 37 | ±1.7 | 1.0 |
| Arginine HCl, | 37 | ±0.6 | 1.0 |
| 0.5% in SIF | | | |
| Arginine free base, | 65 | ±1.1 | 1.8 |
| 0.5% in SIF | | | |
| Trisodium phosphate, | 82 | ±3.7 | 2.2 |
| 0.5% in SIF | | | |

Conclusion: L-Arginine free base as well as trisodium phosphate (Na₃PO₄) show permeation enhancing effects in Caco-2 monolayers.

### Example 7: In Vitro Enzymatic Degradation of Desmopressin by Chymotrypsin

The enzymatic degradation of desmopressin by chymotrypsin was investigated with and without L-arginine free base. Results are shown in Table 6 below.

### Stock Solutions:

Desmopressin: 1 mg/ml in 50 mM phosphate buffer pH 6.5
Buffer pH 9: 0.2 M phosphate buffer, pH=9.2
L-Arginine (free base): 200 mg/ml in 0.2 M phosphate buffer; pH=9.2
Chymotrypsin: 1 mg/ml in 0.2 M phosphate buffer; pH=9.2
Incubation at 37° C. and 400 rpm; 5 hours of incubation
Stop solution: 300 μl 1.25% HCl; analysis via HPLC

TABLE 6

| | | | | | Intact |
|---|---|---|---|---|---|
| | | | L-ARG | | desmopressin |
| | Desmo- | Buffer | free | Chymo- | after 5 hours |
| | pressin | pH 9 | base | trypsin | incubation (%) |
| Negative | 100 μl | 200 μl | — | — | 100% |
| control | | | | | |
| Positive | 100 μl | 100 μl | — | 100 μl | 7% |
| control | | | | | |
| DESMO 1 | 100 μl | — | 100 μl | 100 μl | 48% |

Conclusion: The above results show that L-arginine free base decreases the enzymatic degradation of desmopressin by chymotrypsin.

### Example 8: Inhibition of Intestinal Proteases by a Combination of Trisodium Phosphate and Sodium Decanoate

Simulated intestinal fluid (SIF), pH 7 as well as enzymatically active simulated intestinal fluid containing pancreatin (SIF-P), pH 7 was prepared according to USP guidelines. A stock solution of the peptide PTH(1-34) in SIF, pH 7, with a concentration of 1 mg PTH(1-34) per ml was prepared. The PTH(1-34) stock solution was incubated in the absence and presence of sodium decanoate (010), trisodium phosphate (Na₃PO₄) and a mixture thereof, dissolved in SIF-P (for compositions refer to Table 7). After a defined incubation time at 37° C. and 250 rpm, the samples were directly injected into a HPLC system and analysed with a gradient method based on H₂O containing 0.1% trifluoroacetic acid and acetonitrile containing 0.1% trifluoroacetic acid regarding the PTH(1-34) content.

Results are shown in Table 7. PTH(1-34) was fully degraded in presence of SIF-P within 10 minutes. In addition, PTH(1-34) was fully degraded in presence of C10 and Na₃PO₄. However, no degradation of PTH(1-34) was observed when incubated in presence of a mixture of C10 and Na₃PO₄.

TABLE 7
Recovery of PTH(1-34), expressed as a percentage of the
concentration of PTH(1-34) incubated for 10 minutes at 37° C.
in SIF as a control in presence of various excipients
and excipient mixtures

| | PTH | | | | | Incubation | |
|---|---|---|---|---|---|---|---|
| | stock | | | Na₃PO₄ | C10 | time | % intact |
| Sample | solution | SIF | SIF-P | (mg) | (mg) | (minutes) | PTH(1-34) |
| 1 | 500 μl | 250 μl | — | — | — | 10 | 100 |
| 2 | 500 μl | — | 250 μl | — | — | 10 | 0 |
| 3 | 500 μl | — | 250 μl | — | 10 | 10 | 0 |
| 4 | 500 μl | — | 250 μl | 50 | — | 10 | 0 |
| 5 | 500 μl | — | 250 μl | 50 | 10 | 10 | 101 |

Conclusion: The combination of Na₃PO₄ with sodium decanoate shows surprisingly good inhibition of intestinal proteases.

### Example 9: Pharmacokinetic Profile of Desmopressin Formulations after Administration into Rat Ileum

Desmopressin formulations were dosed into ileum in volume of 0.4 ml/kg and final desmopressin concentration of 0.08 mg/kg to anaesthetised rats (n=5). The anaesthesia was induced with solutions of Hypnorm and Dormicum mixed in the ratio 3:1. After checking of the depth of anaesthesia a 3-5 cm long incision was made in the skin of abdomen. The caecum was exposed and the distal segment of small intestine was pulled out of the abdominal cavity. The intestine was penetrated by the catheter tip and the catheter was inserted downstream into the ileum lumen at a distance of 5 cm from caecum in a spot without faeces, outside the area with accumulated lymphatic tissue and outside the blood vessels and fixed with ligature. The prepared syringes filled with the dosing solution were gradually attached to the inserted catheters. Dosing was performed slowly. Blood was taken from tail vessels at the time points 0, 30, 60, 120 and 240 min after dosing. The desmopressin plasma concentrations were determined using commercial desmopressin EIA kit (Peninsula Laboratories International, USA, cat. no. S-1365.0001). Results are shown in Table 8.

### Compositions:

DESMO-A (=Reference formulation, desmopressin only)
0.2 mg/ml Desmopressin

### DESMO-B

0.2 mg/ml Desmopressin
1 mg/ml Aprotinin

### DESMO-C

0.2 mg/ml Desmopressin
100 mg/ml Tri Sodium Phosphate (Na₃PO₄)

TABLE 8

| | | | | ΔAUC₍₀₋ₜ₎ |
|---|---|---|---|---|
| | pH of final | Cₘₐₓ | | improvement |
| Composition | composition | (ng/ml) | ΔAUC₍₀₋ₜ₎ | factor |
| DESMO-A | 5.7 | 2.7 ± 0.4 | 47 ± 24 | 1.0 |
| DESMO-B | 5.5 | 3.3 ± 1.2 | 229 ± 128 | 4.9 |
| DESMO-C | 11.8 | 4.4 ± 0.5 | 475 ± 62 | 10.1 |

Conclusion: Tri Sodium Phosphate (Na₃PO₄) several fold improved the absorption of desmopressin from rat ileum.

### Example 10: Pharmacokinetic Profile of Desmopressin Formulations after Administration into Rat Ileum

Desmopressin formulations were dosed into ileum in volume of 0.4 ml/kg and final desmopressin concentration of 0.092 mg/kg to anaesthetised rats (n=5). The anaesthesia was induced with solutions of Hypnorm and Dormicum mixed in the ratio 3:1. After checking of the depth of anaesthesia a 3-5 cm long incision was made in the skin of abdomen. The caecum was exposed and the distal segment of small intestine was pulled out of the abdominal cavity. The intestine was penetrated by the catheter tip and the catheter was inserted downstream into the ileum lumen at a distance of 5 cm from caecum in a spot without faeces, outside the area with accumulated lymphatic tissue and outside the blood vessels and fixed with ligature. The prepared syringes filled with the dosing solution were gradually attached to the inserted catheters. Dosing was performed slowly. Blood was taken from tail vessels at the time points 0, 30, 60, 120 and 240 min after dosing. The desmopressin plasma concentrations were determined using commercial desmopressin EIA kit (Peninsula Laboratories International, USA, cat. no. S-1365.0001). Results are shown in Table 9.

### Compositions:

DESMO-A (=Reference formulation, desmopressin only)
0.23 mg/ml Desmopressin

### DESMO-B

0.23 mg/ml Desmopressin
100 mg/ml Mono Sodium Phosphate

### DESMO-C

0.23 mg/ml Desmopressin
100 mg/ml Di Sodium Phosphate

### DESMO-D

0.23 mg/ml Desmopressin
100 mg/ml Tri Sodium Phosphate (Na₃PO₄)

### DESMO-E

0.23 mg/ml Desmopressin
100 mg/ml Tri Sodium Phosphate (Na₃PO₄)
10 mg/ml Sodium caprate (Sodium decanoate)

### DESMO-F

0.23 mg/ml Desmopressin
100 mg/ml Tri Sodium Phosphate (Na₃PO₄)
50 mg/ml Sucrose Laurate

TABLE 9

| | | | | ΔAUC₍₀₋ₜ₎ |
|---|---|---|---|---|
| | pH of final | Cₘₐₓ | | improvement |
| Composition | composition | (ng/ml) | ΔAUC₍₀₋ₜ₎ | factor |
| DESMO-A | 5.6 | 5.0 ± 1.0 | 514 ± 116 | 1.0 |
| DESMO-B | 3.8 | 1.5 ± 0.2 | 87 ± 29 | 0.2 |
| DESMO-C | 8.8 | 2.0 ± 0.4 | 124 ± 52 | 0.2 |
| DESMO-D | 11.8 | 10.9 ± 2.4 | 1 719 ± 490 | 3.3 |
| DESMO-E | 11.7 | 11.7 ± 1.7 | 1 880 ± 268 | 3.6 |
| DESMO-F | 11.7 | 13.3 ± 3.5 | 2 176 ± 551 | 4.2 |

Conclusion: Tri sodium phosphate significantly improved the absorption of desmopressin from rat ileum whereas the mono- and di sodium phosphate did not result in any improvements.

### Example 11: Pharmacokinetic Profile of Octreotide Formulations after Administration into Rat Jejunum

Octreotide formulations were dosed into proximal jejunum in volume of 0.4 ml/kg and final octreotide concentration of 0.4 mg/kg to anaesthetised rats (n=4). The anaesthesia was induced with solutions of Hypnorm and Dormicum mixed in the ratio 3:1. After checking of the depth of anaesthesia a 3-5 cm long incision was made in the skin of abdomen. The caecum was exposed and the small intestine was pulled out of the abdominal cavity up to duodenojejunal flexure. The intestine was penetrated by the catheter tip and the catheter was inserted downstream into the jejunum lumen at a distance of 10±5 cm from the flexure in a spot without faeces, outside the area with accumulated lymphatic tissue and outside the blood vessels and fixed with ligature. The prepared syringes filled with the dosing solution were gradually attached to the inserted catheters. Dosing was performed slowly. Blood was taken from tail vessels at the time points 0, 10, 20, 60 and 120 min after dosing. The octreotide plasma concentrations were determined using commercial octreotide kit (Peninsula Laboratories International, Inc., USA, cat. number S-1342.0001). Results are shown in Table 10 and FIG. 2.

### Compositions:

### OCT005

1 mg/ml Octreotide

### OCT006

1 mg/ml Octreotide
100 mg/ml Tri Sodium Phosphate (Na₃PO₄)

TABLE 10
PK parameters of the two formulations

| | | Δ Octreotide (ng/ml) | | | | | | Δ AUC₍₀₋₁₂₀₎ |
|---|---|---|---|---|---|---|---|---|
| | | 0 | 10 | 20 | 60 | 120 | Δ AUC₍₀₋₁₂₀₎ | Improvement |
| Formulation | | min. | min. | min. | min. | min. | (ng/ml × min) | factor |
| OCT005 | Avg. | 0.0 | 1.9 | 1.3 | 0.6 | 0.2 | 98 | 1.0 |
| | S.E. | ±0.0 | ±0.7 | ±0.4 | ±0.1 | ±0.1 | ±25 | |
| OCT006 | Avg. | 0.0 | 0.4 | 1.4 | 5.6 | 4.6 | 459 | 4.7 |
| | S.E. | ±0.0 | ±0.2 | ±0.5 | ±1.6 | ±0.3 | ±100 | |

### Example 12: Pharmacokinetic Profile of PTH(1-34) Formulation after Administration into Pig Ileum

An in situ gelling matrix tablet containing 2 mg PTH(1-34), 200 mg L-arginine free base, 10 mg polyacrylate (Carbopol) and sodium caprylate was dosed directly into the ileum of a pig. PTH(1-34) plasma levels were analyzed with a commercial High Sensitivity PTH(1-34) ELISA Kit.

Results: The combination of polyacrylate (Carbopol) with arginine results in an in situ gelling matrix system providing prolonged absorption of PTH(1-34), as also shown in FIG. 3.

### Example 13: In Vitro Enzymatic Degradation of Adalimumab by Trypsin

The enzymatic degradation of the antibody adalimumab in simulated intestinal fluid (SIF) at 37° C. containing trypsin was investigated with and without the inhibitor L-arginine free base.

| | 0.5 mg/ml | | | | |
|---|---|---|---|---|---|
| | Adalimumab | | | Incuba- | |
| | in SIF USP | | 1.0 | tion | |
| | stock | SIF- | mg/ml | time | % intact |
| Sample | solution | USP | Trypsin | (min.) | Adalimumab |
| 1 Control | 500 μl | 500 μl | n.a. | 120 | 100% |
| without | | | | | |
| enzyme | | | | | |
| 2 Reference | 500 μl | — | 500 μl | 120 | 82% |
| with | | | | | |
| enzyme | | | | | |
| 3 Enzyme | 500 μl | — | 500 μl + | 120 | 100% |
| and | | | 100 mg | | |
| inhibitor | | | arginine | | |
| | | | base | | |

Conclusion: The above results show that adalimumab is being degraded by trypsin but is fully protected in presence of L-arginine base.

### Example 14: Pharmacokinetic Profile of PTH(1-34) Formulations after Administration into Rat Jejunum

The PTH(1-34) formulations described in the following can be dosed into the jejunum of anaesthetised rats, and PTH(1-34) plasma concentrations can be determined, in analogy to the procedures described in Example 11 above. The improved pharmacokinetic properties of the exemplary formulation according to the present invention, containing PTH(1-34) in combination with trisodium phosphate and sodium caprate, can thus be demonstrated.

### Compositions:

### PTH(1-34)-A

0.2 mg/ml PTH(1-34)
20 mg/ml Sodium Caprate

### PTH(1-34)-B

0.2 mg/ml PTH(1-34)
20 mg/ml Sodium Caprate
100 mg/ml Tri Sodium Phosphate (Na₃PO₄)

### Example 15: Pharmacokinetic Profile of PTH(1-34) Formulations after Oral Administration to Non-Human Primates

Solid oral dosage forms comprising PTH(1-34) have been prepared for in vivo testing in non-human monkeys. The solid oral dosage forms were dosed orally to cynomolgus macaques with a body weight between 5 and 6 kg. Blood was collected at time points 0, 15, 30, 60, 120 and 180 minutes after oral administration. PTH(1-34) plasma concentrations were analysed with a High Sensitivity Human PTH(1-34) ELISA Kit from Immutopics. The pharmacokinetic profiles are shown in FIG. 4.

### Compositions:

### Reference (1)

Size 4 gelatin capsule

### 2 mg PTH(1-34)

### 50 mg Mannitol

### Formulation 2

Size 4 gelatin capsule

### 2 mg PTH(1-34)

### 100 mg L-Arginine

### Formulation 3

Tablet compressed with a compression force of 5.4 kN

### 2 mg PTH(1-34)

### 100 mg L-Arginine

### 100 mg Sucrose Laurate

### Formulation 4

Tablet compressed with a compression force of 5.8 kN

### 2 mg PTH(1-34)

### 100 mg L-Arginine

### 100 mg Sucrose Stearate

### Formulation 5

Tablet compressed with a compression force of 6.9 kN

### 2 mg PTH(1-34)

### 100 mg SNAC

### 50 mg L-Arginine

### 50 mg Sucrose Stearate

### Formulation 6

Tablet compressed with a compression force of 7.8 kN

### 2 mg PTH(1-34)

### 100 mg L-Arginine

### 90 mg Mannitol

### 2.5 mg Carbopol 974P

### SNAC

Gelatin capsule

### 2.5 mg PTH(1-34)

### 100 mg SNAC

Conclusion: Formulations according to the invention comprising arginine resulted in significant oral bioavailability of PTH(1-34) whereas the bioavailability of the control formulation without arginine was neglectable. Formulations comprising arginine also showed improved bioavailability of PTH(1-34) compared to a formulation with only SNAC.

### Example 16: Development of Slow Eroding Tablet Formulations with Semaglutide and L-Arginine

Semaglutide tablets were made with a Korsch EK0 tablet press and their disintegration time in simulated gastric fluid (SGF) at 37° C. was analysed with a disintegration tester according to USP.

### Composition SEMA-A:

### 5 mg Semaglutide

### 300 mg L-Arginine

### 300 mg Sorbitol

### 50 mg Avicel

was compressed with a compression force of 10.4 kN. The tablet disintegrated immediately within a few seconds.

### Composition SEMA-B:

### 5 mg Semaglutide

### 300 mg L-Arginine

### 250 mg Sorbitol

### 50 mg Sucrose Stearate

was compressed with a compression force of 5.5 kN. The tablet showed a prolonged disintegration profile of 5.2 minutes.

### Composition SEMA-C:

### 5 mg Semaglutide

### 300 mg L-Arginine

### 250 mg Sorbitol

### 100 mg Sucrose Stearate

was compressed with a compression force of 8.6 kN. The tablet showed a prolonged disintegration profile of 15.3 minutes.

Conclusion: Tablets with a slow disintegration profile can be prepared by combining arginine and sucrose stearate.

### Example 17: Pharmacokinetic Profile of Semaglutide after Oral Administration in Beagle Dogs

Formulation SEMA-D: Acid resistant hard capsules containing 1.7 mg of semaglutide, 150 mg of sodium caprate and 150 mg of L-arginine free base were dosed orally to overnight fasted beagle dogs (n=3). The intact capsules were dosed orally. Immediately after the administration of the formulation, 10 mL of water was administered to facilitate swallowing. As reference 0.1 mg of semaglutide (Volume of 0.1 ml/dog) was dosed intravenously to beagle dogs (n=3). Approximately 2 mL of blood sample from each dog for the following time points for oral dosing: 0, 1, 2, 4 and 6 hours were collected from the jugular or cephalic or saphenous vein into K₂EDTA containing pre-labeled vacutainer centrifuge tubes. Plasma was obtained by centrifuging blood samples at 5000 g for 5 min at 4° C. within 0.5 h after sampling. The obtained plasma samples were separated into two aliquots and transferred into pre-labeled micro-centrifuge tubes approximately ˜500 μL and were stored at or below −70±10° C. Semaglutide content was analyzed with a commercial semaglutide Elisa Kit.

Results: An absolute oral bioavailability of 3.1% was achieved.

### Example 18: Pharmacokinetic Profile of a Semaglutide Formulation after Administration into Rat Ileum

Formulation SEMA-E (50.5 mg/ml chenodeoxycholate sodium salt and 5.0 mg/ml Na₃PO₄) was dosed into rat ileum in volume of 0.4 ml/kg (final semaglutide concentration 2.02 mg/ml) to anaesthetized rats (n=5). The anaesthesia was induced with Hypnorm/Dormicum mixture. The caecum was exposed and the distal segment of small intestine was pulled out of the abdominal cavity. The intestine was penetrated by the catheter tip and the catheter was inserted downstream into the ileum lumen at a distance of 5 cm from caecum. The pulled segment of small intestine was replaced into the abdominal cavity, 2 ml of sterile saline were flushed over the intestine and the abdominal cavity was closed with metal wound clips. The prepared syringes filled with the dosing solution were gradually attached to the inserted catheters. Dosing was performed slowly. Blood was taken from tail vessels at the time points 0, 30, 60, 120 and 240 min after dosing. 450 μl of blood were drawn from the tail tip into Microtainer Microgard EDTA tubes (Becton Dickinson, USA). Blood samples were centrifuged (4 min, 10,000 g, 4° C.) and approximately 200 μl of plasma were collected. The plasma samples were kept at −20° C. until the semaglutide analysis. The semaglutide plasma concentrations were determined using commercial semaglutide EIA kit (Peninsula Laboratories International, USA, cat. number S-1530.0001).

Results: The administration of the formulation SEMA-E resulted in a high semaglutide plasma concentration with a ΔCₘₐₓ of 787±263 ng/ml and an ΔAUC₍₀₋ₜ₎ of 134910±47574 (ng/ml×min).

### Example 19: In Vitro Enzymatic Degradation of Desmopressin in Presence of L-Lysine

A stock solution of desmopressin (1 mg/ml) in water was prepared. Simulated intestinal fluid containing pancreatin (SIF-P) pH 7, according to the USP was prepared, comprising 1 g of pancreatin in 100 ml of simulated intestinal fluid. The desmopressin stock solution was used to dissolve the lysine salts: L-Lysine base (1) and D-Lysine base (2), each containing 100 mg of lysine per ml as well as 1 mg/ml of desmopressin in water. For the actual degradation experiment, 0.5 ml of the different desmopressin containing stock solutions were incubated for 1 hours at 37° C. and 300 rpm with 0.5 ml of SIF-P (as well as one control, omitting pancreatin). After the incubation period, the enzymatic reaction was stopped by the addition of 1.0 ml of 3.125% HCl in 50% of acetonitrile. Samples were analyzed via HPLC. The desmopressin concentration of the sample omitting pancreatin was used as the 100% value, desmopressin concentrations measured in the other samples were calculated as percentage of intact desmopressin, based on the 100% value. Results are summarized in the Table below.

TABLE
Recovery of intact desmopressin in presence and absence of Lysine

| | | | Peptide |
|---|---|---|---|
| Sample | Lysine | Pancreatin | recovery |
| Reference A | — | No | 100% |
| Reference B | — | Yes | 20% |
| (1) | L-Lysine free | Yes | 58% |
| | base | | |
| (2) | D-Lysine free | Yes | 62% |
| | base | | |

Conclusion: The results show that lysine can partially protect the peptide desmopressin from enzymatic degradation

### Example 20: Oral Formulations with the Antibody Infliximab

Oral formulations with the antibody infliximab were prepared and the final pH was measured, as detailed in the following table. L-arginine improves the solubility of L-tyrosine.

| | 1.0 mg/ml | | | |
|---|---|---|---|---|
| | Infliximab | Enzyme | Visual | |
| Sample | in SIF | inhibitor | inspection | pH |
| 1 | 1000 μl | — | Clear | pH = 7.2 |
| | | | solution | |
| 2 | 1000 μl | 100 mg/ml | Clear | pH = 10.4 |
| | | L-arginine | solution | |
| 3 | 1000 μl | 100 mg/ml | Clear | pH = 10.1 |
| | | L-lysine | solution | |
| 4 | 1000 μl | 100 mg/ml | Cloudy | n.a. |
| | | L-tyrosine | suspension | |
| 4 | 1000 μl | 90 mg/ml | Clear | pH = 10.1 |
| | | L-arginine | solution | |
| | | and | | |
| | | 10 mg/ml | | |
| | | L-tyrosine | | |

### Example 21: Permeation of Semaglutide Through Human Nasal Cells

All permeation studies were performed with RPMI 2650 models after 3 weeks of cultivation. Furthermore, the whole equipment was pre-warmed to 37° C. All steps were performed at 37° C. Prior to permeation, TEER values were measured using EVOM® combined with Endohm® Chamber from World Precision Instruments (WPI, Sarasota, Fla., US).

Afterwards the medium of the RPMI models was replaced by Krebs-Ringer buffer (KRB) from the basal (1500 μL) and apical (500 μL) side to remove medium compounds and to adapt the tissue to the experimental conditions. RPMI models were incubated for 60 min in KRB. Meanwhile sample solution containing excipients and active pharmaceutical ingredients were prepared. Subsequent to the incubation period, TEER values were determined again.

Before permeation, the KRB was removed from both sides of the RPMI models and 1200 μL KRB as acceptor volume were added. Then, 200 μL of the formulations (containing 0.5 mg/ml of Semaglutide each; reference formulation in KRB only, sample formulation in KRB containing 5% of arginine HCl) were applied to the apical surface of each RPMI model. With this step the permeation time was started. After two hours of exposition the acceptor was entirely removed, transferred to Eppendorf tubes and replaced by 1200 μL pre-warmed KRB. Four hours after starting the experiment the total amount of the acceptor was withdrawn again. During permeation the cell culture plates were shaken horizontally with 200 U/min at 37° C. Results are shown in FIG. 5.

Conclusion: Arginine improves the permeation of semaglutide across human nasal cells.

### Example 22: CellTiter 96® AQueous One Solution Cell Proliferation Cytotoxicity Assay (MTS) of Arginine HCl

CellTiter 96® AQueous One Solution Cell proliferation Assay from Promega (Mannheim, Germany) is a colorimetric method for determining the number of viable cells in a cytotoxicity assay. The MTS tetrazolium compound is bioreduced by viable cells in a colored formazan product. The quantity of formazan is directly proportional to the number of living cells and can be measured by absorbance at 490 nm. This assay was performed to determinate cytotoxic effects of Arginine HCl in the concentrations of 2.5% and 5.0% (m/V) on RPMI 2650 cells.

RPMI 2650 cells were seeded onto a 96-well tissue culture test plate at a density of 60,000 cells per well for 24 h. Arginine HCl was dissolved in KRB. Medium was removed from the cells and replaced by 100 μL excipient solutions per well. Cells were incubated with excipient solutions for 0 min, 15 min, 30 min, 60 min and 120 min. Furthermore, cells were incubated with KRB, 1.0% Triton-X solution (V/V) for the same incubation periods as negative and positive controls, respectively. Bacitracin (BAC) served as reference.

The CellTiter 96® AQueous One Solution Cell proliferation Assay was carried out following the manufacturer's protocol. Therefore, the CellTiter 96® AQueous One Solution Reagent was completely thawed. Subsequent to the incubation period 20 μL CellTiter 96® AQueous One Solution Reagent were pipetted into each well of the 96-well assay plate containing 100 μL of the excipient solution. Plates were incubated at 37° C. for 3 h in humidified, 5% CO₂ atmosphere.

After the incubation period the samples were analyzed immediately. Absorbance was recorded at 490 nm and measured by using a microplate reader Infinite® M Plex (Tecan, Switzerland). The results are shown in FIG. 6.

Conclusion: Only a minor decrease in cell viability was observed in presence of arginine HCl in this cell assay and much less cell toxicity was seen as compared to the reference with bacitracin (BAC). Therefore, Arginine HCl can be regarded as a safe excipient for nasal application, even in high concentrations such as 5%.

### Example 23: Transepithelial Electrical Resistance (TEER) Measurement in Presence of Arginine Hydrochloride

The cellZscope from nanoAnalytics (Munster, Germany) was used for continuous evaluation of TEER as a surrogate parameter of tight junction functionality. MDCK cells were grown on 1.12 cm², 1.0 μm transparent filter inserts (ThinCerts™, Greiner Bio-One, Frickenhausen, Germany) with a seeding density of 100,000 cells per well. The medium was changed at cultivation days 3, 4 and 5. TEER measurements were performed on day 5 when cells reached a resistance of approximately 3,500 Ω·cm² starting with the replacement of the growth medium by an equivalent volume of KRB, pH 7.4 (500 μL apical, 1,500 μL basolateral) and cell incubation for 60 min. After this preincubation, 250 μL of KRB was removed from the insert, and the same volume of double-concentrated sample solution (Arginine HCl in KRB) was added to the 250 μL KRB inside the insert to reach the desired concentration. Afterwards, impedance measurements were performed for 180 min with a frequency range of 1 Hz to 100 kHz at 37° C. and 5% CO₂ inside the incubator.

Subsequently, the cells were carefully washed with fresh KRB and incubated in medium, and TEER was monitored up to 24 h in 30 min time intervals after the start of the experiment to evaluate TEER recovery. The results (shown in FIG. 7) are given as the relative TEER reduction, defined as the percentage change of the starting value corrected by the baseline value.

Conclusion: A strong decrease in TEER was observed in presence of arginine, which can be linked to an opening of tight junctions. It has been demonstrated that an opening of tight junctions increases the apical to basolateral transport of hydrophilic drugs, such as peptides and proteins. Moreover, the opening of the tight junctions was fully reversible at both, 2.5% and 5%, leading to the conclusion that the effect is not based on cell toxicity and the tested concentrations are safe for use.

### Example 24: Permeation of Adalimumab Through Human Nasal Cells

All permeation studies were performed with RPMI 2650 models after 3 weeks of cultivation. Furthermore, the whole equipment was pre-warmed to 37° C. All steps were performed at 37° C. Prior to permeation, TEER values were measured using EVOM® combined with Endohm® Chamber from World Precision Instruments (WPI, Sarasota, Fla., US).

Afterwards the medium of the RPMI models was replaced by Krebs-Ringer buffer (KRB) from the basal (1500 μL) and apical (500 μL) side to remove medium compounds and to adapt the tissue to the experimental conditions. RPMI models were incubated for 60 min in KRB. Meanwhile sample solution containing excipients and active pharmaceutical ingredients were prepared. Subsequent to the incubation period, TEER values were determined again.

Before permeation, the KRB was removed from both sides of the RPMI models and 1200 μL KRB as acceptor volume were added. Then, 200 μL of the formulations (containing 2.0 mg/ml of Adalimumab each; reference formulation in KRB only, sample formulation in KRB containing 5% of arginine HCl) were applied to the apical surface of each RPMI model. With this step the permeation time was started. After two hours of exposition the acceptor was entirely removed, transferred to Eppendorf tubes and replaced by 1200 μL pre-warmed KRB. Four hours after starting the experiment the total amount of the acceptor was withdrawn again. During permeation the cell culture plates were shaken horizontally with 200 U/min at 37° C.

The results thus obtained are summarized in the following table:

| | | Mean | S.D. | S.E. | Improvement |
|---|---|---|---|---|---|
| Formulation | Incubation | (μg/ml) | (μg/ml) | (μg/ml) | factor |
| Buffer | 2 hours | 1.3 | 0.6 | 0.3 | — |
| Buffer | 4 hours | 2.2 | 0.7 | 0.4 | — |
| Arg. HCl 5% | 2 hours | 2.2 | 0.7 | 0.4 | 1.7 |
| Arg. HCl 5% | 4 hours | 5.2 | 3.5 | 2.0 | 2.4 |

Conclusion: The presence of 5% Arginine HCl increased the mean apical to basolateral permeation of the antibody adalimumab through human nasal cells.

1. A pharmaceutical composition for transmucosal administration, comprising a peptide or protein drug and an excipient with a pKₐ value of 12 or higher.

2. Use of a peptide or protein drug and an excipient with a pKₐ value of 12 or higher in the preparation of a pharmaceutical composition for transmucosal administration.

3. A method of treating or preventing a disease/disorder, the method comprising transmucosally administering, to a subject in need thereof, a pharmaceutical composition comprising a peptide or protein drug and an excipient with a pKₐ value of 12 or higher.

4. A method of transmucosally delivering a peptide or protein drug, the method comprising transmucosally administering a pharmaceutical composition comprising said peptide or protein drug and an excipient with a pKₐ value of 12 or higher to a subject in need thereof.

5. The pharmaceutical composition of claim 1 or the use of claim 2 or the method of claim 3 or 4, wherein the excipient with a pKₐ value of 12 or higher is selected from arginine free base, EDTA tetrasodium salt, trisodium phosphate, tris(hydroxymethyl)aminomethane, lysine, and calcium hydroxide.

6. The pharmaceutical composition of claim 1 or the use of claim 2 or the method of claim 3 or 4, wherein the excipient with a pKₐ value of 12 or higher is arginine free base or trisodium phosphate.

7. The pharmaceutical composition of claim 1 or the use of claim 2 or the method of claim 3 or 4, wherein the excipient with a pKₐ value of 12 or higher is arginine free base.

8. The pharmaceutical composition of any one of claim 1 or 5 to 7 or the use of any one of claim 2 or 5 to 7 or the method of any one of claims 3 to 7, wherein the peptide or protein drug has a molecular weight of equal to or less than about 50 kDa, preferably a molecular weight of about 1 kDa to about 6 kDa.

9. The pharmaceutical composition of any one of claim 1 or 5 to 7 or the use of any one of claim 2 or 5 to 7 or the method of any one of claims 3 to 7, wherein the peptide or protein drug is selected from insulin, an insulin analog, insulin lispro, insulin PEGlispro, insulin aspart, insulin glulisine, insulin glargine, insulin detemir, NPH insulin, insulin degludec, B29K(N(ε)hexadecanedioyl-γ-L-Glu) A14E B25H desB30 human insulin, B29K(N(ε)octadecanedioyl-γ-L-Glu-OEG-OEG) desB30 human insulin, B29K(N(ε)octadecanedioyl-γ-L-Glu) A14E B25H desB30 human insulin, B29K(N(ε)eicosanedioyl-γ-L-Glu) A14E B25H desB30 human insulin, B29K(N(ε)octadecanedioyl-γ-L-Glu-OEG-OEG) A14E B25H desB30 human insulin, B29K(N(ε)eicosanedioyl-γ-L-Glu-OEG-OEG) A14E B25H desB30 human insulin, B29K(N(ε)eicosanedioyl-γ-L-Glu-OEG-OEG) A14E B16H B25H desB30 human insulin, B29K(N(ε)hexadecanedioyl-γ-L-Glu) A14E B16H B25H desB30 human insulin, B29K(N(ε)eicosanedioyl-γ-L-Glu-OEG-OEG) A14E B16H B25H desB30 human insulin, B29K(N(ε)octadecanedioyl) A14E B25H desB30 human insulin, GLP-1, a GLP-1 analog, an acylated GLP-1 analog, a diacylated GLP-1 analog, a GLP-1 agonist, semaglutide, liraglutide, exenatide, exendin-4, lixisenatide, taspoglutide, albiglutide, dulaglutide, langlenatide, beinaglutide, efpeglenatide, GLP-1(7-37), GLP-1(7-36)NH₂, a dual agonist of the GLP-1 receptor and another receptor, a dual agonist of the GLP-1 receptor and the glucagon receptor, a dual agonist of the GLP-1 receptor and the gastric inhibitory polypeptide receptor, oxyntomodulin, GLP-2, a GLP-2 analog, a GLP-2 agonist, teduglutide, elsiglutide, glucose-dependent insulinotropic polypeptide, a dual GLP-1 analog, a GLP-1R/GCGR dual agonist, a GLP1/glucagon receptor co-agonist, a GLP-1R/GIPR dual agonist, a GLP1/GIP receptor co-agonist, an exendin-4 peptide analog, an exendin-4 derivative, elamipretide, a cyclotide, recombinant factor VIIa, eptacog alfa, amylin, an amylin analog, pramlintide, a somatostatin analog, octreotide, lanreotide, pasireotide, goserelin, buserelin, leptin, a leptin analog, metreleptin, peptide YY, a peptide YY analog, glatiramer, leuprolide, desmopressin, a desmopressin analog, a vasopressin receptor 2 agonist peptide, osteocalcin, an osteocalcin analog or derivative, human growth hormone, a human growth hormone analog, a long-acting human growth hormone, fibroblast growth factor 21, somapacitan, hGH-CTP, an antibody, a glycopeptide antibiotic, a glycosylated cyclic or polycyclic nonribosomal peptide antibiotic, vancomycin, teicoplanin, telavancin, bleomycin, ramoplanin, decaplanin, a cyclotide, bortezomib, cosyntropin, chorionic gonadotropin, menotropin, sermorelin, luteinizing-hormone-releasing hormone, somatropin, calcitonin, calcitonin-salmon, pentagastrin, oxytocin, neseritide, anakinra, enfuvirtide, pegvisomant, dornase alfa, lepirudin, anidulafungin, eptifibatide, interferon alfacon-1, interferon alpha-2a, interferon alpha-2b, interferon beta-1a, interferon beta-1b, interferon gamma-1b, peginterferon alfa-2a, peginterferon alfa-2b, peginterferon beta-1a, fibrinolysin, vasopressin, aldesleukin, an epoetin, epoetin alfa, darbepoetin alfa, epoetin beta, epoetin delta, epoetin omega, epoetin zeta, epoetin theta, methoxy polyethylene glycol-epoetin beta, continuous erythropoietin receptor activator, peglylated epo, albupoetin, an epo-dimer analogue, epo-Fc, carbamylated EPO, synthetic erythropoese protein, the low molecular epo analogue PBI-1402, filgrastim, PEG-filgrastim, interleukin-11, cyclosporine, glucagon, urokinase, viomycin, thyrotropin-releasing hormone, leucine-enkephalin, methionine-enkephalin, substance P, adrenocorticotropic hormone, parathyroid hormone, a parathyroid hormone fragment, teriparatide, PTH(1-31), PTH(2-34), parathyroid hormone-related protein, abaloparatide, linaclotide, carfilzomib, icatibant, ecallantide, cilengitide, a prostaglandin Fla receptor modulator, PDC31, abciximab, ranibizumab, alefacept, romiplostim, anakinra, abatacept, belatacept, and pharmaceutically acceptable salts thereof.

10. The pharmaceutical composition of any one of claim 1 or 5 to 7 or the use of any one of claim 2 or 5 to 7 or the method of any one of claims 3 to 7, wherein the peptide or protein drug is selected from a GLP-1 agonist, semaglutide, liraglutide, exenatide, exendin-4, lixisenatide, taspoglutide, albiglutide, dulaglutide, langlenatide, beinaglutide, efpeglenatide, GLP-1(7-37), GLP-1(7-36)NH₂, a dual agonist of the GLP-1 receptor and another receptor, a dual agonist of the GLP-1 receptor and the glucagon receptor, a dual agonist of the GLP-1 receptor and the gastric inhibitory polypeptide receptor, oxyntomodulin, GLP-2, a GLP-2 agonist, teduglutide, elsiglutide, a somatostatin analog, octreotide, lanreotide, pasireotide, desmopressin, a desmopressin analog, a vasopressin receptor 2 agonist peptide, a parathyroid hormone fragment, teriparatide, PTH(1-31), PTH(2-34), and pharmaceutically acceptable salts thereof.

11. The pharmaceutical composition of any one of claim 1 or 5 to 7 or the use of any one of claim 2 or 5 to 7 or the method of any one of claims 3 to 7, wherein the peptide or protein drug is selected from a GLP-1 agonist, semaglutide, liraglutide, exenatide, exendin-4, lixisenatide, taspoglutide, albiglutide, dulaglutide, langlenatide, beinaglutide, efpeglenatide, GLP-1(7-37), GLP-1(7-36)NH₂, a dual agonist of the GLP-1 receptor and the glucagon receptor, oxyntomodulin, and pharmaceutically acceptable salts thereof.

12. The pharmaceutical composition of any one of claim 1 or 5 to 11 or the use of any one of claim 2 or 5 to 11 or the method of any one of claims 3 to 11, wherein the pharmaceutical composition further comprises a permeation enhancer.

13. The pharmaceutical composition of claim 12 or the use of claim 12 or the method of claim 12, wherein the permeation enhancer is selected from C₈₋₂₀ alkanoyl carnitine, salicylic acid, a salicylic acid derivative, 3-methoxysalicylic acid, 5-methoxysalicylic acid, homovanillic acid, a C₈₋₂₀ alkanoic acid, citric acid, tartaric acid, a fatty acid acylated amino acid, a C₈₋₂₀ alkanoyl sarcosinate, an alkylsaccharide, a C₈₋₁₀ alkylpolysaccharide, n-octyl-beta-D-glucopyranoside, n-dodecyl-beta-D-maltoside, n-tetradecyl-beta-D-maltoside, tridecyl-beta-D-maltoside, sucrose laurate, sucrose laurate, sucrose myristate, sucrose palmitate, sucrose cocoate, sucrose mono-dodecanoate, sucrose mono-tridecanoate, sucrose mono-tetradecanoate, a coco-glucoside, a cyclodextrine, α-cyclodextrin, β-cyclodextrin, γ-cyclodextrin, methyl-β-cyclodextrin, hydroxypropyl β-cyclodextrin, sulfobutylether β-cyclodextrin, N-[8-(2-hydroxybenzoyl)amino]caprylic acid, sodium N-[8-(2-hydroxybenzoyl)amino]caprylate, a sodium N-[8-(2-hydroxybenzoyl)amino]caprylate derivative, a thiomer, a mucoadhesive polymer having a vitamin B partial structure, a calcium chelating compound, ethylenediaminetetraacetic acid, ethylene glycol tetraacetic acid, polyacrylic acid, cremophor EL, chitosan, N,N,N-trimethyl chitosan, benzalkonium chloride, bestatin, cetylpyridinium chloride, cetyltrimethylammonium bromide, a C₂₋₂₀ alkanol, a C₈₋₂₀ alkenol, a C₈₋₂₀ alkenoic acid, dextran sulfate, diethyleneglycol monoethyl ether, 1-dodecylazacyclo-heptan-2-one, caprylocaproyl polyoxylglycerides, ethyl caprylate, glyceryl monolaurate, lysophosphatidylcholine, menthol, a C₈₋₂₀ alkylamine, a C₈₋₂₀ alkenylamine, phosphatidylcholine, a poloxamer, polyethylene glycol monolaurate, polyoxyethylene, polypropylene glycol monolaurate, a polysorbate, cholic acid, a deoxycholate, a chenodeoxycholate, sodium glycocholate, sodium glycodeoxycholate, sodium lauryl sulfate, sodium decyl sulfate, sodium octyl sulfate, sodium laureth sulfate, N-lauryl sarcosinate, decyltrimethyl ammonium bromide, benzyldimethyl dodecyl ammonium chloride, myristyltrimethyl ammonium chloride, dodecyl pyridinium chloride, decyldimethyl ammonio propane sulfonate, myristyldimethyl ammonio propane sulfonate, palmityldimethyl ammonio propane sulfonate, ChemBetaine CAS, ChemBetaine Oleyl, Nonylphenoxypolyoxyethylene, polyoxyethylene sorbitan monolaurate, polyoxyethylene sorbitan monopalmitate, sorbitan monooleate, Triton X-100, hexanoic acid, heptanoic acid, methyl laurate, isopropyl myristate, isopropyl palmitate, methyl palmitate, diethyl sebaccate, sodium oleate, urea, lauryl amine, caprolactam, methyl pyrrolidone, octyl pyrrolidone, methyl piperazine, phenyl piperazine, Carbopol 934P, glyccyrhetinic acid, bromelain, pinene oxide, limonene, cineole, octyl dodecanol, fenchone, menthone, trimethoxy propylene methyl benzene, a cell-penetrating peptide, KLAKLAK, polyarginine, oligoarginine, octa-arginine, penetratin, a penetratin analog, PenetraMax, HIV-1 Tat, transportan, macrogol-15-hydroxystearate, Solutol HS 15, CriticalSorb, a taurocholate, a taurodeoxycholate, a sulfoxide, decyl methyl sulfoxide, dimethyl sulfoxide, cyclopentadecalactone, 8-(N-2-hydroxy-5-chloro-benzoyl)-amino-caprylic acid, N-(10-[2-hydroxybenzoyl]amino)decanoic acid, dodecyl-2-N,N-dimethylamino propionate, D-α-tocopheryl polyethylene glycol-1000 succinate, arginine, and pharmaceutically acceptable salts thereof;
and further wherein said fatty acid acylated amino acid is preferably selected from sodium lauroyl alaninate, N-dodecanoyl-L-alanine, sodium lauroyl asparaginate, N-dodecanoyl-L-asparagine, sodium lauroyl aspartic acid, N-dodecanoyl-L-aspartic acid, sodium lauroyl cysteinate, N-dodecanoyl-L-cysteine, sodium lauroyl glutamic acid, N-dodecanoyl-L-glutamic acid, sodium lauroyl glutaminate, N-dodecanoyl-L-glutamine, sodium lauroyl glycinate, N-dodecanoyl-L-glycine, sodium lauroyl histidinate, N-dodecanoyl-L-histidine, sodium lauroyl isoleucinate, N-dodecanoyl-L-isoleucine, sodium lauroyl leucinate, N-dodecanoyl-L-leucine, sodium lauroyl methioninate, N-dodecanoyl-L-methionine, sodium lauroyl phenylalaninate, N-dodecanoyl-L-phenylalanine, sodium lauroyl prolinate, N-dodecanoyl-L-proline, sodium lauroyl serinate, N-dodecanoyl-L-serine, sodium lauroyl threoninate, N-dodecanoyl-L-threonine, sodium lauroyl tryptophanate, N-dodecanoyl-L-tryptophane, sodium lauroyl tyrosinate, N-dodecanoyl-L-tyrosine, sodium lauroyl valinate, N-dodecanoyl-L-valine, sodium lauroyl sarcosinate, N-dodecanoyl-L-sarcosine, sodium capric alaninate, N-decanoyl-L-alanine, sodium capric asparaginate, N-decanoyl-L-asparagine, sodium capric aspartic acid, N-decanoyl-L-aspartic acid, sodium capric cysteinate, N-decanoyl-L-cysteine, sodium capric glutamic acid, N-decanoyl-L-glutamic acid, sodium capric glutaminate, N-decanoyl-L-glutamine, sodium capric glycinate, N-decanoyl-L-glycine, sodium capric histidinate, N-decanoyl-L-histidine, sodium capric isoleucinate, N-decanoyl-L-isoleucine, sodium capric leucinate, N-decanoyl-L-leucine, sodium capric methioninate, N-decanoyl-L-methionine, sodium capric phenylalaninate, N-decanoyl-L-phenylalanine, sodium capric prolinate, N-decanoyl-L-proline, sodium capric serinate, N-decanoyl-L-serine, sodium capric threoninate, N-decanoyl-L-threonine, sodium capric tryptophanate, N-decanoyl-L-tryptophane, sodium capric tyrosinate, N-decanoyl-L-tyrosine, sodium capric valinate, N-decanoyl-L-valine, sodium capric sarcosinate, N-decanoyl-L-sarcosine, sodium oleoyl sarcosinate, sodium N-decylleucine, sodium stearoyl glutamate, sodium myristoyl glutamate, sodium lauroyl glutamate, sodium cocoyl glutamate, sodium cocoyl glycinate, sodium N-decyl leucine, sodium cocoyl glycine, sodium cocoyl glutamate, sodium lauroyl alaninate, N-dodecanoyl-L-alanine, sodium lauroyl asparaginate, N-dodecanoyl-L-asparagine, sodium lauroyl aspartic acid, N-dodecanoyl-L-aspartic acid, sodium lauroyl cysteinate, N-dodecanoyl-L-cysteine, sodium lauroyl glutamic acid, N-dodecanoyl-L-glutamic acid, sodium lauroyl glutaminate, N-dodecanoyl-L-glutamine, sodium lauroyl glycinate, N-dodecanoyl-L-glycine, sodium lauroyl histidinate, N-dodecanoyl-L-histidine, sodium lauroyl isoleucinate, N-dodecanoyl-L-isoleucine, sodium lauroyl leucinate, N-dodecanoyl-L-leucine, sodium lauroyl methinoninate, N-dodecanoyl-L-methionine, sodium lauroyl phenylalaninate, N-dodecanoyl-L-phenylalanine, sodium lauroyl prolinate, N-dodecanoyl-L-proline, sodium lauroyl serinate, N-dodecanoyl-L-serine, sodium lauroyl threoninate, N-dodecanoyl-L-threonine, sodium lauroyl tryptophanate, N-dodecanoyl-L-tryptophane, sodium lauroyl tyrosinate, N-dodecanoyl-L-tyrosine, sodium lauroyl valinate, N-dodecanoyl-L-valine, N-dodecanoyl-L-sarcosine, sodium capric alaninate, N-decanoyl-L-alanine, sodium capric asparaginate, N-decanoyl-L-asparagine, sodium capric aspartic acid, N-decanoyl-L-aspartic acid, Sodium capric cysteinate, N-decanoyl-L-cysteine, sodium capric glutamic acid, N-decanoyl-L-glutamic acid, sodium capric glutaminate, N-decanoyl-L-glutamine, sodium capric glycinate, N-decanoyl-L-glycine, sodium capric histidinate, N-decanoyl-L-histidine, sodium capric isoleucinate, N-decanoyl-L-isoleucine, sodium capric leucinate, N-decanoyl-L-leucine, leucine, sodium capric methioninate, N-decanoyl-L-methionine, sodium capric phenylalaninate, N-decanoyl-L-phenylalanine, sodium capric prolinate, N-decanoyl-L-proline, sodium capric serinate, N-decanoyl-L-serine, sodium capric threoninate, N-decanoyl-L-threonine, sodium capric tryptophanate, N-decanoyl-L-tryptophane, sodium capric tyrosinate, N-decanoyl-L-tyrosine, sodium capric valinate, N-decanoyl-L-valine, sodium capric sarcosinate, sodium oleoyl sarcosinate, and pharmaceutically acceptable salts thereof.

14. The pharmaceutical composition of claim 12 or the use of claim 12 or the method of claim 12, wherein the permeation enhancer is selected from sodium caprylate, sodium caprate, sodium laurate, sucrose laurate, sucrose laurate, sodium stearate, EDTA, polyacrylic acid, and sodium N-[8-(2-hydroxybenzoyl)amino]caprylate.

15. The pharmaceutical composition of any one of claim 1 or 5 to 14 or the use of any one of claim 2 or 5 to 14 or the method of any one of claims 3 to 14, wherein the constitution of the pharmaceutical composition is such that, if the pharmaceutical composition is added to 10 ml of 0.1 M aqueous sodium bicarbonate solution, the pH of the solution will be higher than pH 9.

16. The pharmaceutical composition of any one of claim 1 or 5 to 15 or the use of any one of claim 2 or 5 to 15 or the method of any one of claims 3 to 15, wherein the pharmaceutical composition comprises the excipient with a pKₐ value of 12 or higher in an amount of about 1 mg to about 1000 mg per dosage unit, preferably in an amount of about 50 mg to about 500 mg per dosage unit.

17. The pharmaceutical composition of any one of claim 1 or 5 to 16 or the use of any one of claim 2 or 5 to 16 or the method of any one of claims 3 to 16, wherein the peptide or protein drug is physically separated from the excipient with a pKₐ value of 12 or higher within the pharmaceutical composition.

18. The pharmaceutical composition of any one of claim 1 or 5 to 17 or the use of any one of claim 2 or 5 to 17 or the method of any one of claims 3 to 17, wherein the pharmaceutical composition comprises particles of the excipient with a pKₐ value of 12 or higher, wherein said particles are coated with a protective coating that separates the excipient with a pKₐ value of 12 or higher from the peptide or protein drug, and wherein the protective coating is preferably made of glucose, maltodextrin, or HPMC.

19. The pharmaceutical composition of any one of claim 1 or 5 to 18 for use as a medicament, wherein said pharmaceutical composition is to be administered transmucosally.

20. The pharmaceutical composition of any one of claim 1 or 5 to 18 for use in treating or preventing a disease/disorder, wherein said pharmaceutical composition is to be administered transmucosally.

21. The pharmaceutical composition of claim 11 for use in treating or preventing diabetes, obesity, or non-alcoholic fatty liver disease, wherein said pharmaceutical composition is to be administered transmucosally.

22. The pharmaceutical composition for use according to any one of claims 19 to 21 or the use of any one of claim 2 or 5 to 18 or the method of any one of claims 3 to 18, wherein said pharmaceutical composition is to be administered orally.

23. The pharmaceutical composition for use according to any one of claims 19 to 21 or the use of any one of claim 2 or 5 to 18 or the method of any one of claims 3 to 18, wherein said pharmaceutical composition is to be administered oromucosally.

24. The pharmaceutical composition for use according to any one of claims 19 to 21 or the use of any one of claim 2 or 5 to 18 or the method of any one of claims 3 to 18, wherein said pharmaceutical composition is to be administered nasally.

25. The pharmaceutical composition of any one of claim 1 or 5 to 18 for use in treating or preventing an intestinal disease/disorder, wherein said pharmaceutical composition is to be administered orally.

26. The pharmaceutical composition for use according to claim 22 or 25 or the use of claim 22 or the method of claim 22, wherein said pharmaceutical composition is a solid composition.


SEQUENCE LISTING (2 sequences)

SEQ ID NO: 1 - protein, 31 aa, Artificial Sequence
  GLP-1(7-37)
HAEGTFTSDVSSYLEGQAAKEFIAWLVKGRG

SEQ ID NO: 2 - protein, 40 aa, Artificial Sequence
  Exendin-4
HGEGTFITSDLSKQMEEEAVRLFIEWLKNGGPSSGAPPPS